(19) United States
(12) Patent Application Publication
(10) Pub. No.: US 2024/0173329 A1
(43) Pub. Date: May 30, 2024

(54) BELVARAFENIB FOR USE IN TREATMENT OF BRAIN CANCERS

(71) Applicants: Genentech, Inc., South San Francisco, CA (US); Hoffmann-La Roche Inc., Little Falls, NJ (US); Hanmi Pharm. Co., Ltd., Seoul (KR)

(72) Inventors: Joanne Adamkewicz, South San Francisco, CA (US); Michael John Dolton, Sydney (AU); Shiva Malek, South San Francisco, CA (US); Piia Thomas, South San Francisco, CA (US); Jennifer Eng-Wong, South San Francisco, CA (US); Yibing Yan, South San Francisco, CA (US); Young Hoon KIM, Seoul (KR); Young Gil AHN, Seoul (KR); Yu Yon KIM, Seoul (KR)

(21) Appl. No.: 18/549,622

(22) PCT Filed: Mar. 8, 2022
(86) PCT No.: PCT/US22/19273

§ 371 Date: Sep. 8, 2023

Related U.S. Application Data
(60) Provisional application No. 63/158,798, filed on Mar. 9, 2021.

(51) Int. Cl. A61K 31/519; A61K 31/4523; A61P 35/04

(52) U.S. Cl. CPC A61K 31/519; A61K 31/4523; A61P 35/04

(57) ABSTRACT

Provided are methods for the use of belvarafenib to treat brain cancer including metastatic brain cancer carrying a BRAF mutation, a NRAS mutation, a KRAS mutation, or a combination thereof.

### CROSS REFERENCE TO RELATED APPLICATION

This application claims priority to U.S. Provisional Application Ser. No. 63/158,798 filed on Mar. 9, 2021. The entire text of that provisional application is incorporated by reference into this application

### FIELD OF THE DISCLOSURE

The field of the disclosure relates generally to treatment of cancers in the brain, and more specifically relates to treatment of cancers in which RAF-kinase inhibition is effective.

### BACKGROUND

Based on data from the NIH, the rate of new cases of brain and other nervous system cancer in 2017 was 6.4 per 100,000, the death rate was 4.4 per 100,000, and about 0.6 percent of the population will be diagnosed with brain and other nervous system cancer at some point during their lifetime. Further, the 5-year survival rate for the period from 2010 to 2016 was only about 33%.

Problematically, the efficacy of a brain cancer therapy drug is dependent on the ability of the drug to cross the brain capillary wall, which forms the blood-brain barrier (BBB), and enter the brain in a therapeutically effective amount. By some estimates, less than 2% of small molecule drug actually cross the BBB in any significant amount. (Pardridge, The Blood-Brain Barrier: Bottleneck in Brain Drug Development, NeuroRx. 2005 Jan. 2(1): 3-14).

Glioblastoma is the most common brain tumor originating in the brain with about 15,000 new cases diagnosed each year, and the average survival rate is about 11 to 15 months.

Metastatic brain tumors, originating from cancer formed elsewhere in the body, are the most common brain tumor among adults with an estimated 200,000 and 300,000 new cases diagnosed each year. Many such brain tumors arise from spread of cancers—such as melanoma—that are caused by malfunctions of the MAPK signaling pathway arising from BRAF and NRAS mutations. Certain pediatric brain tumors such as pilocytic astrocytomas, are also believed to be caused by mutations in MAPK proteins such as BRAF (see, e.g., Faulkner et al., J. Neuropathol. Exp. Neurol., 74: 867-872, (2015); Penman, et al., Frontiers in Oncology, 5:54, 2015; and Kurani, Child's Nervous System, 35:1525-1536, (2019)).

Over the past decade, BRAF inhibitors, either alone or in combination with MEK inhibitors, have set a new milestone in the treatment of patients with BRAF V600 mutation melanoma, which accounts for about 50% of melanomas. Nevertheless, highly aggressive metastases to the brain are associated with poor outcomes in melanoma patients, and therefore still require better treatment. Also, to date, no targeted therapy has been developed for patients with NRAS mutations, which account for about 20% of melanoma.

A need therefore exists for improved chemotherapy treatments for brain cancers including metastatic brain cancers having NRAS and RAF mutations.

### BRIEF DESCRIPTION

In some aspects, the present disclosure is directed to a method of treating metastatic melanoma comprising administering to a subject in need thereof an amount of belvarafenib effective to treat the metastatic melanoma, wherein the site of metastasis is within the subject's brain.

In some aspects, the present disclosure is directed to a method of treating brain cancer. The method comprises: (i) administering to a subject in need thereof a therapeutically effective amount of belvarafenib, or a pharmaceutically acceptable salt thereof, to treat the brain cancer; (ii) wherein administration of the belvarafenib inhibits the growth and viability of brain cancer cells in said subject; and (iii) wherein the brain cancer is characterized by a mutated MAPK signaling pathway.

In some other aspects, the present disclosure is directed to a method of treating brain cancer involving a combination therapy. The method comprises: (i) administering to a subject in need thereof a therapeutically effective amount of belvarafenib, or a pharmaceutically acceptable salt thereof, (ii) administering to the subject an effective amount of a MEK inhibitor, or a pharmaceutically acceptable salt thereof; (iii) wherein administration of the belvarafenib and the MEK inhibitor inhibits the growth and viability of brain cancer cells in said subject; and (iv) wherein the brain cancer is characterized by a mutated MAPK signaling pathway.

Belvarafenib was found to be able to accumulate in the brain of mice and rats following oral administration. The exposure of belvarafenib in the brain was similar to that in plasma (approximately 100% brain to plasma ratio). This level of brain penetration of belvarafenib differs significantly from currently approved BRAF inhibitors that are known to have low levels of brain penetration. Belvarafenib showed excellent anti-tumor activity in an orthotopic brain tumor model using melanoma cells. Belvarafenib significantly increased the overall survival of mice implanted intracranially with BRAF^{V600E} A375SM melanoma cells. Furthermore, the data presented herein also demonstrates that belvarafenib has therapeutic potential to treat NRAS mutation melanoma patients.

As belvarafenib is a pan-Raf inhibitor, any brain-metastasised tumor originating from Raf malignancy could be treated.

### BRIEF DESCRIPTION OF THE DRAWINGS

FIG. 1 is a collection of representative bioluminescence images of mice under evaluation in an A375SM_Luc cell brain metastasis mouse model of the present disclosure.

FIG. 2 is a plot of a Kaplan-Meier survival curve of mice under evaluation in an A375SM_Luc cell brain metastasis mouse model of the present disclosure.

### DETAILED DESCRIPTION

### Belvarafenib

Belvarafenib is a potent and selective pan-RAF kinase inhibitor showing strong inhibition activities for BRAF WT, BRAF^{V600E} and CRAF. It has previously been reported that belvarafenib strongly inhibits the growth of various cancer cell lines harboring BRAF, NRAS or KRAS mutations, and demonstrates remarkable anticancer efficacy in preclinical animal models.

Belvarafenib is disclosed in PCT application WO 2013/100632, has the chemical name 4-amino-N-(1-((3-chloro-2-fluorophenyl)amino)-6-methylisoquinolin-5-yl)thieno[3,2-d]pyrimidine-7-carboxamide (referred to herein as Formula (I)), and has the following chemical structure:

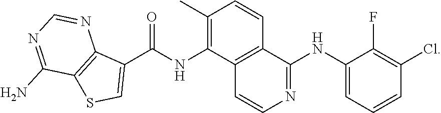 [SMILES: Cc1ccc2c(Nc3cccc(Cl)c3F)nccc2c1NC(=O)c1csc2c(N)ncnc12]

Belvarafenib is a highly potent and selective type II RAF dimer inhibitor (a pan-RAF inhibitor) that provides for selective inhibition of BRAF and CRAF isoforms. In contrast with BRAF^{V600}-selective monomer inhibitors, belvarafenib does not activate the MAPK pathway in non-BRAF^{V600} mutant cells, but instead sustains the suppression of MAPK signaling by inhibiting BRAF and CRAF dimers, and results in reduced cell proliferation and increased antitumor activity in both BRAF^{V600} and RAS-mutant tumors.

The RAF kinase family, which consists of three subtypes (A-RAF, B-RAF, C-RAF), is a key component of the MAPK signaling pathway downstream of RAS. Mutations in RAF genes, particularly BRAF at codon V600, have been identified in various cancers, including malignant melanoma, colorectal, thyroid, and lung cancers. See Davies H, Bignell G R, Cox C, et al., “Mutations of the BRAF gene in human cancer”, Nature 417:949-54, 200. The BRAF V⁶⁰⁰ mutations enable BRAF to signal as a monomer, thereby constitutively activating the downstream MAPK signaling pathway

RAS genes are the most frequently mutated oncogenes in human cancer. Among the RAS isoforms, KRAS is the most frequently mutated (86%), followed by NRAS (11%), which is predominantly mutated in cutaneous melanoma (28%). See: Cox A D, Fesik S W, Kimmelman A C, et al, “Drugging the undruggable RAS: Mission possible?”, Nat Rev Drug Discov 13:828-51, 2014; Hilmi Kodaz, Osman Kostek, Muhammet Bekir Hacioglu, et al., “Frequency of RAS Mutations (KRAS, NRAS, HRAS) in Human Solid Cancer”, EJMO 1:1-7, 2017; and Cancer Genome Atlas N, “Genomic Classification of Cutaneous Melanoma”, Cell 161:1681-96, 2015.

The discovery of BRAF monomer inhibitors, such as, vemurafenib, dabrafenib, and encorafenib, has led to notable advances in the treatment of patients with BRAF^{V600}-mutant tumors; nevertheless, the durability of treatment response has been limited due to a variety of resistance mechanisms including BRAF amplification, BRAF splice variants and RAS mutations, that largely converge on BRAF dimerization, and resistance to BRAF^{V600} monomer therapies. See Sullivan R J, Flaherty K T, “Resistance to BRAF-targeted therapy in melanoma” Eur J Cancer 49:1297-304, 2013.

Belvarafenib inhibits phosphorylation of MEK and ERK in the MAPK pathway in BRAF- or RAS-mutant melanoma, NSCLC, and CRC cell lines. Belvarafenib has been demonstrated to inhibit the growth of BRAF- or RAS-mutant melanoma, NSCLC, CRC, and thyroid cancer cell lines in vitro.

Belvarafenib is a potent and selective inhibitor of the RAF kinases including BRAF^{V600E} mutant (IC50=7 nM), BRAF wild type (IC50=41 nM), and RAF-1 (CRAF) (IC50=2 nM) in vitro. When tested in a panel of 189 kinase assays, belvarafenib showed inhibitory activity against 7 other receptor tyrosine kinases (RTKs) (colony stimulating factor 1 receptor (CSF1R), formerly McDonough feline sarcoma (FMS) homolog, discoidin domain receptor tyrosine kinase 1 (DDR1), discoidin domain receptor tyrosine kinase 2 (DDR2), EPHA2, EPHA7, EPHA8, and EPHB2) with >90% inhibition at 1 μM.

The in vitro antitumor effects of belvarafenib have translated into efficacy in various mouse xenograft models. Belvarafenib shows dose-dependent inhibition of tumor growth in mouse xenograft models as a monotherapy against BRAF- and NRAS-mutant melanoma, against KRAS mutant non-small cell lung cancer (NSCLC), and against BRAF mutant colorectal cancer (CRC) mouse xenograft models.

### Belvarafenib Clinical Data

Belvarafenib has by now been shown in clinical trials to provide safe and efficacious therapy against a number of cancers.

For instance, a completed, open-label, Phase Ia, dose-escalation investigated several doses and schedules of belvarafenib in patients with solid tumors harboring mutations in BRAF, KRAS, or NRAS genes. Efficacy was analyzed for 67 of 72 subjects who had at least 1 post-baseline tumor assessment. Best overall response rate (BORR) was 8.96% (6/67 subjects), objective response rate (ORR) was 4.48% (3/67 subjects) with partial response (PR) as confirmed best overall response (2 subjects with melanoma and a subject with gastrointestinal stromal tumor). Disease control was observed in 50.57% (34/67) of subjects treated with belvarafenib 100 mg QD dose level or above. Fifty-nine (88.06%) subjects developed an event (progression of disease or death), all of which were reported as progressive disease (PD). In addition, median progression-free survival was 11.53 weeks and the 95% confidence interval for the median was [7.12 weeks, 13.38 weeks). In updated results, BORR was 10.45% (7/67 subjects) and the 95% exact confidence interval was [4.30%, 20.35%]; ORR remains at 4.48% (3/67 subjects). In addition, subgroup re-analysis for BRAF-mutant melanoma subjects showed 7.69% (1/13 subjects) of BORR, DCR, median PFS, and time to progression were not changed in total subjects. Median DOR was elevated to 30.18 weeks in 800 mg BID group and 23.99 weeks in total group including DOR of a subject which is 100.29 weeks.

In another open-label, Phase Ib, dose-expansion study, belvarafenib was evaluated at a dose of 450 mg BID in patients with solid tumors harboring mutations in BRAF, KRAS, or NRAS genes. Efficacy was analyzed for 59 of 63 subjects who had at least 1 dose of belvarafenib after enrollment and had at least 1 post-baseline tumor assessment. BORR was 11.86% (7/59 subjects), ORR was 6.78% (4/59 subjects) with PR as confirmed best overall response (3 subjects with melanoma and a subject with CRC). Disease control was observed in 35.59% (21/59) of subjects. Fifty (84.75%) of 59 subjects developed an event (progression of disease or death), all of which were reported as PD except 1 death case. In addition, median progression-free survival (PFS) was 7.83 weeks and the 95% confidence interval for the median was [7.26 weeks, 8.26 weeks]. Median duration of response (DOR) of a total response in this study was 15.66 weeks from 7 responders. Among them, 2 BRAF-mutant melanoma responders showed 22.49 weeks of median DOR.

In another Phase I, single dose, randomized, crossover relative bioavailability and food effect study in healthy subjects, the influence of a formulation change from the Phase I to Phase II tablet on belvarafenib exposure was evaluated. A total of 18 healthy subjects were enrolled in the study and received the following randomized treatments: one 150-mg and one 50-mg Phase I tablet in a fed state, two 100-mg Phase II tablets in a fed state, or two 100-mg Phase II tablets in a fasted state, with an 18-day washout between treatments. There was a positive effect of food on belvarafenib exposure in the fed state compared to the fasted state. Belvarafenib exposure, Cₘₐₓ and AUC_{0-inf}, were increased by approximately 2.2- and 2.8-fold, respectively, when belvarafenib was administered in the fed state compared to the fasted state in healthy subjects at a 200 mg single dose. No serious adverse events, adverse events of special interest, or deaths were reported in the study.

### Belvarafenib Effective Against Cancers of the Brain

In accordance with the present disclosure, it has been discovered that belvarafenib effectively crosses the BBB and is an effective chemotherapy drug for the treatment of cancers in the brain.

As used herein, “blood brain barrier” or “BBB” refers to a highly selective semipermeable border of endothelial cells that prevents solutes in the circulating blood from non-selectively crossing into the extracellular fluid of the central nervous system. See Daneman “The blood-brain barrier”, Cold Spring Harbor Perspectives in Biology 7 (1) 2015.

Based on experimental evidence to date, it is believed that belvarafenib effectively penetrates into the brain (as shown in animal studies) and that belvarafenib is not a substrate of the two most express efflux inhibitors, P-gp and BCRP. As is known in the art, if a drug is a substrate for one or both of these efflux inhibitors, the drug is usually effluxed back into the blood and will generally have low or no relevant brain penetration. For instance, the RAF inhibitor drug encorafenib is a P-gp and a BCRP efflux substrate and provides for a brain to plasma ratio of only about 0.004 (see Pharmacol Res 2018; 129: 414-23). Further, the RAF inhibitor drug vemurafenib is a P-gp and a BCRP efflux substrate and provides for a brain to plasma ratio of only from about 0.004 to about 0.01 (see Pharmacol Exp Ther 2012; 342: 33-40). Yet further, the RAF inhibitor drug dabrafenib is a P-gp and a BCRP efflux substrate and provides for a brain to plasma ratio of only from about 0.02 to about 0.04 (see Pharmacol Exp Ther 2013; 344: 655-64). In contrast, Belvarafenib is not a P-gp and a BCRP efflux substrate and provides for a brain to plasma ratio of from about 0.8 to about 1.2. The discovery of the high brain penetration of belvarafenib may provide a therapeutic option for brain cancers and metastatic brain cancers carrying an MAPK mutation.

The most common causes of brain metastasis in adults with their approximate frequency are below:

| Indication | Cancer subtype | Frequency (%) |
|---|---|---|
| Melanoma ¹ | | 44% |
| NSCLC ² | Stage I/II | 27.5 |
| | Stage III | 19.7 |
| | Stage IV | 47.1 |
| Breast cancer ³ | HR⁺/HER2⁻ | 38.8 |
| | HR⁺/HER2⁺ | 15.7 |
| | HR⁻/HER2⁻ | 10.7 |
| | TNBC | 18.7 |
| Colorectal cancer ⁴ | — | 4.4 |
| HCC 5 | — | 37.1~90.0 |

Abbreviations
NSCLC: non-small cell lung cancer,
HER2: human epidermal growth factor receptor 2,
HR: hormone receptor,
⁺denotes positive,
⁻denotes negative,
TNBC: triple-negative breast cancer,
HCC: hepatocellular carcinoma

### REFERENCES

1. Cancer 2011, 117(8), pp 1687-96 (doi.org/10.1002/cncr.25634)
2. Neuro Oncol. 2015, 17(1), pp 122-128. (doi: 10.1093/neuonc/nou099)
3. Cancer Manag Res, 2018, 10, pp 5329-5338 (doi: 10.2147/CMAR.S176763)
4. Tumori Journal, 2019, 105(5), 427-433 (doi.org/10.1177/0300891618765541)
5. Oncotarget, 2017, 8, pp 25814-25829 (doi.org/10.18632/oncotarget.15730)

In accordance with the present disclosure, it has been discovered in animal studies that belvarafenib effectively crosses the BBB. It is known and accepted in the art that rodent models of the experiments of the present disclosure are reliably predictive of the effect in humans. It is therefore believed that belvarafenib will effectively cross the BBB in humans. It is further believed that the present examples, where human tumor cells were transplanted into the brains of nude mice and where the tumors were shown to regress in response to oral belvarafenib therapy by means of a luciferase assay, point to the likelihood of translatability of the therapy to humans.

It is likely that a metastatic brain tumor arises from a form of cancer that has advanced to stage IIIb or more at the time of detection.

The present invention can be applicable to brain-specific cancers, as well as some that depend on MAPK pathway. In the latter case, brain metastasis is a main cause of death, meaning that ways of effectively targeting metastatic brain tumors can be effective in prolonging survival. In situations of brain metastasis, it is normal to try chemotherapy and/or radiation therapy and, if unsuccessful, more targeted therapies such as combinations of RAF and MEK inhibitors can be used.

Based on brain pharmacokinetic experiments of the present disclosure, it is believed that belvarafenib, administered in doses that have been shown in clinical trials to be safe and well tolerated, effectively crosses the BBB in therapeutic amounts. One such indicative pharmacokinetic property is the brain to plasma concentration ratio, B/P. The belvarafenib B/P ratio may be calculated from the area under the curve (AUCₗₐₛₜ) for the brain in units of ng·hour/g of brain tissue and from the AUCₗₐₛₜ for plasma in units of ng·hour/mL. In accordance with the present disclosure, the belvarafenib B/P is about 0.3, about 0.4, about 0.5, about 0.6, about 0.7, about 0.8, about 0.9, about 1.0, about 1.1, about 1.2, about 1.3, about 1.4, about 1.5, about 1.6, or about 1.7 or more, and any range constructed therefrom, for instance from about 0.3 to about 1.7, from about 0.7 to about 1.6, or from about 0.8 to about 1.5. The time to maximum belvarafenib brain concentration (Tₘₐₓ) is about 4 hours, about 5 hours, about 6 hours, about 7 hours, about 8 hours, about 9 hours, or about 10 hours, and any range constructed therefrom, for instance from about 4 hours to about 10 hours, from about 5 hours to about 9 hours, or from about 6 hours to about 8 hours. The half-life (t_{1/2}) of belvarafenib in the brains is about 3 hours, about 4 hours, about 5 hours, about 6 hours, about 7 hours, about 8 hours, about 9 hours, about 10 hours, about 11 hours, or about 12 hours, and any range constructed therefrom, for instance from about 3 hours to about 12 hours, from about 4 hours to about 11 hours, or from about 5 hours to about 10 hours. The mean residence time in the brain (MRTₗₐₛₜ) is about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, or about 20 hours, and any range constructed therefrom, for instance from about 7 hours to about 20 hours, from about 9 hours to about 18 hours, or from about 10 hours to about 17 hours.

Belvarafenib brain AUCₗₐₛₜ and peak brain concentration (Cₘₐₓ) varies with the dose. At a dose of 15 mg per kg body weight, the AUCₗₐₛₜ in units of ng·hour/g of brain tissue is about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, or about 100,000 or more, and any range constructed therefrom, for instance from about 25,000 to about 100,000, from about 50,000 to about 90,000, or from about 65,000 to about 80,000. At a dose of 15 mg per kg body weight, the Cₘₐₓ in units of ng belvarafenib per gram of brain tissue is about 1,000, about 1,500, about 2,000, about 2,500, about 3000, about 3,500, about 4,000, about 4,500, about 5,000, about 5,500, about 6,000, about 6,500, about 7,000, about 7,500, or about 8,000 or more, and any range constructed therefrom, for instance from about 1,000 to about 8,000, from about 2,000 to about 7,000, or from about 3,000 to about 6,000.

### Cancers Treatable

In some aspects, the cancer is metastatic melanoma, and the metastatic melanoma is treated by administering to a subject in need thereof a therapy comprising an amount of belvarafenib effective to treat the metastatic melanoma, wherein the site of metastasis is within the subject's brain.

In some aspects, the cancer is brain cancer, and the brain cancer is treated by: (i) administering to a subject in need thereof a therapeutically effective amount of belvarafenib, or a pharmaceutically acceptable salt thereof, to treat the brain cancer; (ii) wherein administration of the belvarafenib inhibits the growth and viability of brain cancer cells in said subject; and (iii) wherein the brain cancer is characterized by a mutated MAPK signaling pathway.

In some aspects, the cancer is brain cancer, and the brain cancer is treated by: (i) administering to a subject in need thereof a therapeutically effective amount of belvarafenib, or a pharmaceutically acceptable salt thereof, (ii) administering to the subject an effective amount of a MEK inhibitor, or a pharmaceutically acceptable salt thereof; (iii) wherein administration of the belvarafenib and the MEK inhibitor inhibits the growth and viability of brain cancer cells in said subject; and (iv) wherein the brain cancer is characterized by a mutated MAPK signaling pathway.

In some aspects, the cancer is brain cancer and a pharmaceutical composition comprising a therapeutically effective amount of belvarafenib, or a pharmaceutically acceptable salt thereof, is provided for use in the treatment of the brain cancer. In some such aspects, the brain cancer is characterized by a mutated MAPK signaling pathway. In any of such aspects, the administration of belvarafenib inhibit the growth and viability of the brain cancer cells.

In some aspects, the cancer is brain cancer and a pharmaceutical composition comprising a therapeutically effective amount of belvarafenib, or a pharmaceutically acceptable salt thereof, is provided for use in the treatment of the brain cancer. In such aspects, the pharmaceutical composition is administered in combination with a therapeutically effective amount of a MEK inhibitor, or a pharmaceutically acceptable salt thereof, as described elsewhere herein. In some such aspects, the brain cancer is characterized by a mutated MAPK signaling pathway. In any of such aspects, the administration of the combination of belvarafenib and the MEK inhibitor inhibits the growth and viability of the brain cancer cells.

In any of the various aspects of the disclosure, the brain cancer may be a (i) cancer that originates in the brain, such as for instance and without limitation, glioblastoma, (ii) a brain tumor arising in pediatric patients, or (iii) a RAS mutant cancer that has metastasized to the brain including, without limitation, melanoma, non-small cell lung cancer (NSCLC), colorectal (CRC) cancer such as colorectal adenocarcinoma, lung adenocarcinoma, head and neck squamous cell carcinoma, and pancreatic ductal adenocarcinoma. Other such cancers can include breast, bladder urothelial carcinoma, gallbladder, nephroblastoma, gastrointestinal stromal tumor (GIST), prostate, myeloid leukemia, multiple myeloma, thyroid, biliary, adenocarcinoma, choriocarcinoma, and sarcoma. Patients suffering from combinations of any of the foregoing may also be treatable.

In some aspects, the cancer carries a RAF mutation.

In some aspects, the cancer carries a BRAF^{V600E} mutation.

In some aspects, the cancer carries a non-canonical BRAF mutation such as a RAF dimer Class2/fusion mutation, or a class 3 mutation.

In some aspects, the cancer carries one or more of a G13, G12, or Q61 NRAS-mutation.

In some aspects, the cancer has at least one mutation selected from a NRAS^{G12D} mutation, a NRAS^{Q61K} mutation, a NRAS^{Q61R} mutation, a NRAS^{G12R} mutation, and a NRAS^{G12C} mutation, and has metastasized to the brain. In some aspects the cancer is a melanoma carrying a NRAS mutation selected from Q61R, Q61H, Q61K, Q61L, and combinations thereof.

In some aspects, the cancer carries at least one mutation selected from a KRAS^{G12V} mutation, a KRAS^{G12D} mutation, a KRAS^{G12C} mutation, a KRAS^{G12R} mutation, and a KRAS^{Q61H} mutation, and has metastasized to the brain. In some aspects, the cancer is a colorectal adenocarcinoma having a KRAS mutation selected from G12V, G12C, G12D, and combinations thereof. In some aspects, the cancer is a pancreatic ductal adenocarcinoma having a KRAS mutation selected from G12V, G12R, G12D, and combinations thereof. In some aspects, the cancer is a lung adenocarcinoma having a KRAS mutation selected from G12V, G12C, G12D, and combinations thereof.

In some such aspects, the brain cancer has two mutations, such as a BRAF mutation and a NRAS mutation, or a BRAF mutation and a KRAS mutation. In one aspect, the cancer carries a BRAF^{V600E} mutation and a NRAS^{Q61L} mutation.

In some aspects, the cancer has at least one mutation selected from a BRAF^{V600E} mutation, a KRAS^{G12V} mutation, a KRAS^{G12D} mutation, a KRAS^{G12C} mutation, a KRAS^{Q61H} mutation, a NRAS^{G12D} mutation, a NRAS^{Q61K} mutation, a NRAS^{Q61R} mutation, a NRAS^{Q61H} mutation, a NRAS^{Q61L} mutation, and a NRAS^{G12C} mutation.

In some aspects, the cancer is selected from sarcoma carrying a KRAS^{G12V} mutation, melanoma carrying a NRAS^{G12D} mutation, melanoma carrying a NRAS^{Q61K} mutation, melanoma carrying a NRAS^{Q61R} mutation, melanoma carrying a NRAS^{Q61H} mutation melanoma carrying a NRAS^{Q61L} mutation, melanoma carrying a NRAS^{G12C} mutation, gallbladder cancer carrying a KRAS^{G12D} mutation, CRC carrying a KRAS^{G12C} mutation, CRC carrying a KRAS^{G12V} mutation, CRC carrying a KRAS^{Q61H} mutation, CRC carrying a KRAS^{G12D} mutation, bladder cancer carrying a KRA^{G12D} mutation, bladder cancer carrying a KRAS^{G12V} mutation, and combinations thereof.

In some aspects, the cancer is sarcoma carrying a KRAS^{G12V} mutation, melanoma carrying a NRAS^{Q61R} mutation, melanoma carrying a NRAS^{Q61H} mutation, gallbladder cancer carrying a KRAS^{G12D} mutation, CRC carrying a KRAS^{G12C} mutation, CRC carrying a KRAS^{G12V} mutation, CRC carrying a KRAS^{G12D} mutation, bladder cancer carrying a KRAS^{G12D} mutation, bladder cancer carrying a KRAS^{G12V} mutation, and combinations thereof.

In some aspects, the cancer is selected from melanoma carrying a NRAS^{Q61L} mutation, melanoma carrying a NRAS^{Q61H} mutation, melanoma carrying a NRAS^{Q61K} mutation, melanoma carrying a NRAS^{Q61R} mutation, and combinations thereof.

In some aspects, the brain cancer is present in pediatric patients, and is selected from tumors having a BRAF^{V600E} mutation or a KIAA1549-BRAF fusion oncogene. In one aspect, the tumor is a glioma such as a pilocytic astrocytoma. Other gliomas having one or more such BRAF mutations, include: pilomyxoid astrocytoma, anaplastic astrocytoma, glioblastoma multiforme, diffuse fibrillary astrocytoma, pleomorphic xanthoastrocytoma, desmoplastic infantile astrocytoma, and ganglioglioma.

### Dosing

The subject may be suitably treated with about 2.5 mg belvarafenib, or a pharmaceutically acceptable salt thereof, per kg body weight per day, about 5 mg/kg per day, about 7.5 mg/kg per day, about 10 mg/kg per day, about 12.5 mg/kg per day, about 15 mg/kg per day, about 17.5 mg/kg per day, about 20 mg/kg per day, about 22.5 mg/kg per day, or about 25 mg/kg per day, and any range constructed therefrom, such as from about 2.5 mg/kg per day to about 25 mg/kg per day, from about 7.5 mg/kg per day to about 20 mg/kg per day, or from about 10 mg/kg per day to about 15 mg/kg per day.

The belvarafenib dose may range from a dose sufficient to elicit a response to the maximum tolerated dose. For instance, and without being bound to any particular dose, the daily dose may suitably be 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, about 1350 mg, about 1400 mg, about 1450 mg, or about 1500 mg and any rage constructed therefrom, such as from 100 mg to 1300 mg, from 200 mg to 1300 mg, from 600 mg to 1300 mg, from 700 mg to 1200 mg, or from 800 mg to 1000 mg. Belvarafenib can be dosed once per day, twice per day, three times per day, or four times per day, subject to the foregoing daily dose ranges.

In some aspects, belvarafenib is dosed twice per day. In one such aspect, belvarafenib may be dosed at 250 mg BID, 300 mg BID, 350 mg BID, 400 mg BID, 450 mg BID, or 500 mg BID. Dosing may be done with or without food. The dosing schedule may suitably be every day of a 28-day schedule, or 21 or more days of a 28-day schedule.

### Forms of Belvarafenib

Belvarafenib may suitably be in the form of stereoisomers, geometric isomers and tautomers, and solvates, metabolites, isotopes, pharmaceutically acceptable salts, or prodrugs thereof. In some particular aspects, belvarafenib is a pharmaceutically acceptable salt thereof.

As used herein, the term “pharmaceutically acceptable salts” refers to those salts which retain the biological effectiveness and properties of the free bases or free acids, and which are not biologically or otherwise undesirable. Exemplary acid salts of belvarafenib include, but are not limited, to sulfate, citrate, acetate, oxalate, chloride, bromide, iodide, nitrate, phosphate, acid phosphate, lactate, salicylate, acid citrate, tartrate, tartrate, ascorbate, succinate, maleate, fumarate, gluconate, glucuronate, formate, benzoate, glutamate, methanesulfonate “mesylate”, ethanesulfonate, benzenesulfonate, and p-toluenesulfonate. In some aspects, the salt is selected from group consisting of the bis-hydrochloride salt, the bis-hydrogensulfate salt, the bis-p-toluenesulfonate salt, the bis-ethanesulfonate salt, and the bis-methanesulfonate salt. In some aspects, the salt is the bis-hydrochloride salt or the bis-methanesulfonate salt. In one aspect, the salt is the bis-methanesulfonate salt.

Belvarafenib may suitably be either in amorphous or crystalline forms. In some aspects the salt is crystalline. In some such aspects, the salt is the bis-methanesulfonate salt. In some particular aspects, the bis-methanesulfonate salt is characterized by a powder X-ray diffraction (PXRD) pattern having one, two, three, four, five, six, seven, eight, nine or ten peaks, three or more peaks, or five or more peaks selected from those at diffraction angle 2θ±0.2° values of 5.6°, 7.1°, 7.6°, 11.4°, 15.1°, 15.4°, 16.6°, 18.2°, 20.4°, 21.5°, 22.3°, 22.7°, 23.1°, 24.4°, 24.9° and 25.6°, when irradiated with a Cu-Kα light source. In some aspects the salt is the bis-hydrochloride salt. In some particular aspects, the bis-hydrochloride salt is polymorph Form I characterized by a powder X-ray diffraction pattern having three or more peaks selected from those at diffraction angle 2θ values of 5.89°±0.2°, 7.77°±0.2°, 8.31°±0.2°, 11.80°±0.2°, 16.68°±0.2°, 23.22°±0.2°, 23.69°±0.2°, 26.89°±0.2°, 27.51°±0.2°, and 29.53°±0.2°, when irradiated with a Cu-Kα light source. The solid form (crystalline or amorphous) may suitably be determined by PXRD recorded in a D8 ADVANCE made by BRUKER AXS in Germany, operating at 25° C. and at 40.0 KV and 100 mA, using Cu Kα (1.54056 Å) line and rotation.

Belvarafenib, and other actives within the scope of the present disclosure, may suitably be formulated with one or more pharmaceutically acceptable carriers, adjuvants, and/or excipients and in the form of a capsule, tablet (pill), powder, syrup, dispersion, suspension, emulsion, solution, or the like. Non-limiting examples of suitable liquid carriers include water; saline; aqueous dextrose; glycols; ethanol; oils including those of petroleum, animal, vegetable or synthetic origin; and combinations thereof. Non-limiting examples of suitable pharmaceutical adjuvants/excipients include starch, cellulose, polyvinylpyrrolidone, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. Belvarafenib may also be suitably formulated with additional conventional pharmaceutical additives such as preservatives, stabilizing agents, wetting or emulsifying agents, salts for adjusting osmotic pressure, buffers and the like. Such compositions will, in any event, contain an effective amount of belvarafenib so as to prepare the proper dosage form for proper administration to the subject.

Belvarafenib may be suitably administered to the subject orally.

### Function and Action of Belvarafenib

In any of the various aspects of the disclosure, the belvarafenib therapy may result in one or more of: (i) inhibition of brain cancer metastases; (ii) reduction in brain cancer metastases size; (iii) reduction in brain cancer metastases number; (iv) reduction of number of brain cancer cells; (v) reduction of brain cancer cell viability; and (vi) inhibition of brain cancer cell growth.

As used herein “inhibit” and “inhibition” refer to a decrease in the activity of the target enzyme, as compared to the activity of that enzyme in the absence of the inhibitor. In some aspects, the terms “inhibit” and “inhibition” means a decrease in activity of at least about 5%, at least about 10%, at least about 20%, at least about 25%, at least about 50%, at least about 60%, at least about 70%, at least about 80%, at least about 90%, or at least about 95%. In other aspects, inhibit and inhibition mean a decrease in activity of about 5% to about 25%, about 25% to about 50%, about 50% to about 75%, or about 75% to 100%. In some aspects, inhibit and inhibition mean a decrease in activity of about 95% to 100%, e.g., a decrease in activity of 95%, 96%, 97%, 98%, 99%, or 100%. Such decreases can be measured using a variety of techniques that would be recognizable by one of skill in the art.

As used herein, “reduce” and “reduction” refer to a decrease in the indicated metric, such as size, number, and viability, as compared to that metric in the absence of the chemotherapy. In some aspects, the terms “reduce” and “reduction” mean a decrease in the metric of at least about 5%, at least about 10%, at least about 20%, at least about 25%, at least about 50%, at least about 60%, at least about 70%, at least about 80%, at least about 90%, or at least about 95%. In other aspects, reduce and reduction mean a decrease in the metric of about 5% to about 25%, about 25% to about 50%, about 50% to about 75%, or about 75% to 100%. In some aspects, reduce and reduction mean a decrease in the metric of about 95% to 100%, e.g., a decrease in activity of 95%, 96%, 97%, 98%, 99%, or 100%. Such decreases can be measured using a variety of techniques that would be recognizable by one of skill in the art.

Combination Therapy with MEK Inhibitors

In some aspects, at least one additional therapy may be administered with the belvarafenib therapy. In some such aspects, the at least one additional therapy is a chemotherapeutic agent.

In some such aspects, the additional therapy is a MEK inhibitor drug. Examples of MEK inhibitors within the scope of the present disclosure include cobimetinib, trametinib, binimetinib, selumetinib, pimasertib, refametinib, PD-0325901 and BI-847325, and pharmaceutically acceptable salts thereof.

In some particular aspects of the disclosure, the MEK inhibitor is cobimetinib or a pharmaceutically acceptable salt thereof (e.g., Cotellic®). Cobimetinib is a reversible, potent, and highly selective inhibitor of MEK1 and MEK2 (central components of the RAS/RAF/MEK/ERK (MAPK)) pathway and has single agent anti-tumor activity in multiple human cancer models. Cobimetinib has the CAS Registry Number 1168091-68-6, is of the chemical name S)[3,4-difluoro-2-(2-fluoro-4-iodophenylamino)phenyl][3-hydroxy-3-(piperidin-2-yl]azetidin-1-yl) methanone, is of the below structure:

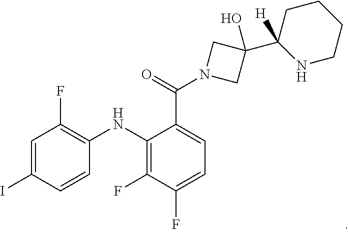 [SMILES: O=C(c1ccc(F)c(F)c1Nc1ccc(I)cc1F)N1CC(O)([C@@H]2CCCCN2)C1]

Cotellic® is the fumarate salt of cobimetinib. Cobimetinib is described in U.S. Pat. Nos. 7,803,839 and 8,362,002, each of which is incorporated by reference in its entirety.

Cobimetinib inhibits proliferation of a variety of human tumor cell lines through inhibition of MEK1 and MEK2. In addition, cobimetinib inhibits ERK phosphorylation in xenograft tumor models and stimulates apoptosis. Cobimetinib accumulates in tumor xenografts and remains at high concentrations in the tumor after plasma concentrations have declined. The activity of cobimetinib to inhibit ERK1 phosphorylation is more closely correlated with its concentration in tumor tissue than in plasma; in general, there is a good correlation between reduced ERK1 phosphorylation and efficacy in tumor xenograft models. Tumor regression has been observed in several human tumor xenograft models. This regression was dose dependent with up to 100% regression at the highest doses tested. The models studied include CRC, malignant melanoma, breast carcinoma, and lung carcinoma.

The pharmacokinetics of cobimetinib administered as a single agent have been characterized in cancer patients following oral administration after single and multiple dosing in Study MEK4592g which included evaluation of a cobimetinib dose of 60 mg per day in patients who harbored a BRAF, NRAS, or KRAS mutation. Overall 6 patients (all of whom had melanoma; 6.2%) had a confirmed partial response (PR), 28 patients (28.9%) had stable disease (SD), and 40 patients (41.2%) had progressive disease. Out of the 14 colorectal cancer (CRC) patients, all patients experienced progressive disease (PD). In Stage III of Study MEK4592g, 18 patients were accrued, and best overall response was assessed for 14 of 18 patients. Four patients (22.2%) had SD as their best overall response, and 2 patients (11.1%) had unconfirmed tumor responses.

Cobimetinib has a moderate rate of absorption (median time to maximum concentration [tₘₐₓ] of 1 to 3 hours) and a mean terminal half-life (t_{1/2}) of 48.8 hours (a range of 23.1 to 80 hours). Cobimetinib binds to plasma proteins (95%) in a concentration-independent manner. Cobimetinib exhibits linear pharmacokinetics in the dose range of 0.05 mg/kg (approximately 3.5 mg/kg for 70 kg adult) to 80 mg and the absolute bioavailability was determined to be 45.9% (90% CI. 39.74%, 53.06%) in study MEK4952g in healthy subjects. Cobimetinib pharmacokinetics are not altered when administered in the fed state compared with administration in the fasted state in healthy subjects. Since food does not alter cobimetinib pharmacokinetics, cobimetinib can be administered with or without food. The proton pump inhibitor rabeprazole appears to have a minimal effect on cobimetinib pharmacokinetics, whether administered in the presence or absence of a high-fat meal compared with cobimetinib administration alone in the fasted state. Thus, increase in gastric pH does not affect cobimetinib pharmacokinetics, indicating it is not sensitive to alterations in gastric pH.

Cobimetinib salts, crystalline forms and prodrugs are within the scope of the present disclosure. Cobimetinib, preparative methods, and therapeutic uses are disclosed in International Publication Numbers WO 2007/044515, WO 2014/027056 and WO 2014/059422, each of which is incorporated herein by reference. For instance, in some aspects of the present disclosure, the MEK inhibitor is crystalline hemifumarate cobimetinib polymorph Form A.

MEK inhibitor (e.g., cobimetinib) doses within the scope of the present disclosure are from about 20 mg to about 100 mg, from about 40 mg to about 80 mg, or about 60 mg of the MEK inhibitor per day. In particular embodiments, the MEK inhibitor is cobimetinib, and is dosed at about 60 mg, about 40 mg or about 20 mg.

The MEK inhibitor is suitably administered once daily. In some aspects, the MEK inhibitor is administered once daily for 21 consecutive days of a 28-day treatment cycle. In some aspects, the MEK inhibitor is administered once daily on days 1 to 21 or on days 3 to 23 of a 28-day treatment cycle.

### EXAMPLES

The Institutional Animal Care and Use Committee of the Hanmi Research Center approved the animal study protocols of examples 1 to 3.

### Example 1: Brain Distribution of Belvarafenib in Mice

Example 1 evaluated pharmacokinetic profile and brain distribution of belvarafenib in mice after oral administration.

The mouse strain was ICR (CD-1®) supplied by Orient Bio Inc., Korea. The mice were male and were 8 weeks of age at the start of dosing, and had a body weight range of 30±1 gram.

The mice were kept in conventional animal lab cages for 5 days for acclimation before the start of the experiment. The cages were polysulfone 1291H (W425×D266×H185 mm, Techniplast, Italy). Twelve mice were housed in each cage at a temperature of 22±2° C., a relative humidity of 50±20%, a ventilation frequency of 10-15 times/h, a 12 hour light/dark cycle, a light intensity of 150-300 Lux, at least weekly cage replacement. The mice were fed Picolab Rodent diet (5053, Lab Diet, USA) with abundant tap water.

The mice were dosed with belvarafenib dihydrochloride, 99.6% purity, that was stored at room temperature. Dosing was based on active ingredient free base and was corrected for assay and water content. The dosing vehicle was DMSO (5%), cremophore EL (5%), and deionized water (90%). Belvarafenib for dosing (28.08 mg) was dissolved in 14.40 mL of the vehicle for a final concentration of 1.950 mg/mL.

In a single one day dosing regimen, mice were fasted overnight. The body weight of the mice was measured on the day of treatment, from which dosing volumes were set. The mice were then dosed with 10 mL/kg of the belvarafenib solution thereby providing an active dose of 15 mg/kg. The mice were fed 4 hours post-dose.

The general clinical signs were observed during the pre- and post-dosing periods. No clinical signs were observed.

Blood (0.3 mL) was collected from the retro-orbital plexus at 0.5, 1, 2, 4, 7, 24, 48, and 72 hours after belvarafenib administration. Plasma was obtained by centrifugation at 12,000 rpm for 2 minutes (Eppendorf) and then harvested and stored at −20° C. (Panasonic, MDF-U334-PK) until analysis.

After bloodletting of mice at each time point, the brain was collected immediately without perfusion. Brain tissue was washed one to two times with saline to remove surface blood. The brains were then trimmed. The collected brain tissue was accurately weighed and mixed with 4-fold saline based on brain weight (a final dilution factor of 5-fold). Each brain sample was then homogenized and placed in a 1.5 mL tube and stored at −20° C. (Panasonic, MDF-U334-PK) until analysis.

Plasma and brain of untreated animals were similarly obtained for use as a blank matrix.

Plasma and brain concentrations of belvarafenib determined by analysis using LC-MS/MS (Waters UPLC H-Class/Xevo TQ (Waters USA)).

Pharmacokinetic parameters of belvarafenib were calculated from plasma and brain concentration-time data by a non-compartmental method using Phoenix™ WinNonlin 8.1 (Certara, USA). The peak plasma and brain concentration (Cₘₐₓ) and the corresponding time (Tₘₐₓ) were directly obtained from the raw data. The area under the plasma curve (AUCₗₐₛₜ) was obtained by linear-log trapezoidal summation. Other PK parameters such as AUC from dosing time extrapolated to infinity (AUC_{inf}), half-life (t_{1/2}) and mean residence time (MTRₗₐₛₜ) were calculated using WinNonlin. The plasma and brain concentrations of belvarafenib were presented as MEAN±S.D. The brain to plasma (B/P) ratio of Belvarafenib was calculated by dividing AUCₗₐₛₜ in the brain by AUCₗₐₛₜ in the plasma.

The mean (±S.D.) plasma and brain concentration-time profiles (AUC), Cₘₐₓ, Tₘₐₓ, t_{1/2}, MRT and B/P ratio in mice of belvarafenib after oral administration at a dose of 15 mg/kg is presented in Table 1.

TABLE 1
Pharmacokinetic parameters of belvarafenib in mice after oral
administration of 15 mg/kg belvarafenib (3 data/time point)

| | | |
|---|---|---|
| Parameter | Plasma | Brain |

| | | | | |
|---|---|---|---|---|
| AUCₗₐₛₜ | 84264.0 | ng · h/mL | 78259.6 | ng · h/g |
| AUC_{inf} | 84347.9 | ng · h/mL | 78297.5 | ng · h/g |
| Cₘₐₓ | 4782.1 | ng/mL | 5238.8 | ng/g |

| | | |
|---|---|---|
| Tₘₐₓ (h) | 4.0 | 7.0 |
| T_{1/2} (h) | 6.6 | 6.3 |
| MRTₗₐₛₜ (h) | 14.3 | 11.8 |

| | |
|---|---|
| B/P ratio | 0.929 |

The mean (±S.D.) plasma and brain concentration-time profiles (AUC), Cₘₐₓ, Tₘₐₓ, and B/P ratio in mice of belvarafenib after oral administration at a dose of 30 mg/kg is presented in Table 2.

TABLE 2
Pharmacokinetic parameters of belvarafenib in mice after oral
administration of 30 mg/kg belvarafenib (3 data/time point)

| | | |
|---|---|---|
| Parameter | Plasma | Brain |

| | | | | |
|---|---|---|---|---|
| AUCₗₐₛₜ | 132721.0 | ng · h/mL | 100735.8 | ng · h/g |
| AUC_{inf} | 133431.0 | ng · h/mL | 100848.0 | ng · h/g |
| Cₘₐₓ | 6211.4 | ng/mL | 6099.0 | ng/g |

| | | |
|---|---|---|
| Tₘₐₓ (h) | 7.0 | 7.0 |

| | |
|---|---|
| B/P ratio | 0.8 |

The results of Example 1 show that following oral administration of belvarafenib at a dose of 15 mg/kg, AUCₗₐₛₜ and Cₘₐₓ in plasma were 84264.0 ng·h/mL and 4782.1 ng/mL, respectively. Plasma Tₘₐₓ and half-life were 4.0 h and 6.6 h, respectively. AUCₗₐₛₜ and Cₘₐₓ in brain were 78259.6 ng·h/g and 5238.8 ng/g, respectively. Brain Tₘₐₓ and half-life were 7.0 h and 6.3 h, respectively. The results of Example 1 show that following oral administration of belvarafenib at a dose of 30 mg/kg, AUCₗₐₛₜ and Cₘₐₓ in plasma were 132731.0 ng·h/mL and 6211.4 ng/mL, respectively. Plasma Tₘₐₓ was 7.0 hours. AUCₗₐₛₜ and Cₘₐₓ in brain were 100735.8 ng·h/g and 6099.0 ng/g, respectively. Brain Tₘₐₓ was 7.0 h. The data show that belvarafenib was slowly distributed to the brain with slightly later Tₘₐₓ than in plasma. The exposure in brain and plasma of belvarafenib was similar with AUCₗₐₛₜ and Cₘₐₓ of about 1-fold difference. The B/P ratio based on AUCₗₐₛₜ of belvarafenib at 15.0 mg/kg and 30.0 mg/kg was 0.929 and 0.8, respectively, in mice, indicating high distribution in mouse brain. Therefore, the example shows high permeability of belvarafenib through the blood-brain barrier.

### Example 2: Brain Distribution of Belvarafenib in Rats

Example 2 evaluated pharmacokinetic profile and brain distribution of belvarafenib in rats after oral administration.

The rat strain was SD supplied by Orient Bio Inc., Korea. The rats were male and were 7 weeks of age at the start of dosing, and had a body weight range of 230.1±6.4 grams.

The rats were kept in conventional animal lab cages for 5 days for acclimation before the start of the experiment. The cages were polysulfone 1291H (W425×D266×H185 mm, Techniplast, Italy). Three rats were housed in each cage at a temperature of 22±2° C., a relative humidity of 50±20%, a ventilation frequency of 10-15 times/h, a 12 hour light/dark cycle, a light intensity of 150-300 Lux, at least weekly cage replacement. The mice were fed Picolab Rodent diet (5053, Lab Diet, USA) with abundant tap water.

The rats were dosed with belvarafenib dihydrochloride, 99.6% purity, that was stored at room temperature. Dosing was based on active ingredient free base and was corrected for assay and water content. The dosing vehicle was DMSO (10%), PEG 400 (50%), and deionized water (40%). Belvarafenib for dosing (169.51 mg) was dissolved in 86.93 mL of the vehicle for a final concentration of 1.950 mg/mL.

In a single one day dosing regimen, rats were fasted overnight. The body weight of the rats was measured on the day of treatment, from which dosing volumes were set. The rats were then dosed with 10 mL/kg of the belvarafenib solution thereby providing an active dose of 15 mg/kg. The rats were fed 4 hours post-dose.

The general clinical signs were observed during the pre- and post-dosing periods. No clinical signs were observed.

Blood (0.3 mL) was collected from the abdominal artery at 0.5, 1, 2, 4, 7, 24, 48, and 72 hours after belvarafenib administration. Plasma was obtained by centrifugation at 12,000 rpm for 2 minutes (Eppendorf) and then harvested and stored at −20° C. (Panasonic, MDF-U334-PK) until analysis.

After bloodletting of rats at each time point, the brain was collected immediately without perfusion. Brain tissue was washed one to two times with saline to remove surface blood. The brains were then trimmed. The collected brain tissue was accurately weighed and mixed with 4-fold saline based on brain weight (a final dilution factor of 5-fold). Each brain sample was then homogenized and placed in a 1.5 mL tube and stored at −20° C. (Panasonic, MDF-U334-PK) until analysis.

Plasma and brain of untreated animals were similarly obtained for use as a blank matrix.

Plasma and brain concentrations of belvarafenib determined by analysis using LC-MS/MS (Waters UPLC H-Class/Xevo TQ (Waters USA)).

Pharmacokinetic parameters of belvarafenib were calculated from plasma and brain concentration-time data by a non-compartmental method using Phoenix™ WinNonlin 8.1 (Certara, USA). The peak plasma and brain concentration (Cₘₐₓ) and the corresponding time (Tₘₐₓ) were directly obtained from the raw data. The area under the plasma curve (AUCₗₐₛₜ) was obtained by linear-log trapezoidal summation. Other PK parameters such as AUC from dosing time extrapolated to infinity (AUC_{inf}), half-life (t_{1/2}) and mean residence time (MTRₗₐₛₜ) were calculated using WinNonlin. The plasma and brain concentrations of belvarafenib were presented as MEAN±S.D. The brain to plasma (B/P) ratio of Belvarafenib was calculated by dividing AUCₗₐₛₜ in the brain by AUCₗₐₛₜ in the plasma.

The mean (±S.D.) plasma and brain concentration-time profiles (AUC), Tₘₐₓ, t_{1/2}, MRT and B/P ratio in rats of belvarafenib after oral administration at a dose of 15 mg/kg is presented in Table 3.

TABLE 3
Pharmacokinetic parameters of belvarafenib in rats after oral
administration of 15 mg/kg belvarafenib (3 data/time point)

| | | |
|---|---|---|
| Parameter | Plasma | Brain |

| | | | | |
|---|---|---|---|---|
| AUCₗₐₛₜ | 49418.4 | ng · h/mL | 67954.7 | ng · h/g |
| AUC_{inf} | 50614.3 | ng · h/mL | 68350.0 | ng · h/g |
| Cₘₐₓ | 2132.9 | ng/mL | 3914.3 | ng/g |

| | | |
|---|---|---|
| Tₘₐₓ (h) | 4.0 | 7.0 |
| T_{1/2} (h) | 12.5 | 9.0 |
| MRTₗₐₛₜ (h) | 18.0 | 15.5 |

| | |
|---|---|
| B/P ratio | 1.375 |

The mean (±S.D.) plasma and brain concentration-time profiles (AUC), Cₘₐₓ, Tₘₐₓ, and B/P ratio in rats of belvarafenib after oral administration at a dose of 30 mg/kg is presented in Table 4.

TABLE 4
Pharmacokinetic parameters of belvarafenib in rats after oral
administration of 30 mg/kg belvarafenib (3 data/time point)

| | | |
|---|---|---|
| Parameter | Plasma | Brain |

| | | | | |
|---|---|---|---|---|
| AUCₗₐₛₜ | 97988.0 | ng · h/mL | 113670.1 | ng · h/g |
| AUC_{inf} | 100034.5 | ng · h/mL | 115222.3 | ng · h/g |
| Cₘₐₓ | 4151.7 | ng/mL | 4876.0 | ng/g |

| | | |
|---|---|---|
| Tₘₐₓ (h) | 7.0 | 4.0 |

| | |
|---|---|
| B/P ratio | 1.2 |

The results of Example 2 show that following oral administration of belvarafenib at a dose of 15 mg/kg, AUCₗₐₛₜ and Cₘₐₓ in plasma were 49418.4 ng·h/mL and 2132.9 ng/mL, respectively. Plasma Tₘₐₓ and half-life were 4.0 h and 12.5 h, respectively. AUCₗₐₛₜ and Cₘₐₓ in brain were 67954.7 ng·h/g and 3914.3 ng/g, respectively. Brain Tₘₐₓ and half-life were 7.0 h and 9.0 h, respectively. The results of Example 2 show that following oral administration of belvarafenib at a dose of 30 mg/kg, AUCₗₐₛₜ and Cₘₐₓ in plasma were 97988.0 ng·h/mL and 4151.7 ng/mL, respectively. Plasma Tₘₐₓ was 7.0 h. AUCₗₐₛₜ and Cₘₐₓ in brain were 113670.1 ng·h/g and 4876.0 ng/g, respectively. Brain Tₘₐₓ was 7.0 h. The exposure in the brain was higher than that in plasma. The B/P ratio based on AUCₗₐₛₜ of belvarafenib was 1.375 and 1.2 for doses of 15 mg/kg and 30 mg/kg, respectively, in rats, indicating high distribution in rat brain. Therefore, the example shows high permeability of belvarafenib through the blood-brain barrier.

### Example 3: Interaction of Belvarafenib with the Human BCRP and MDR1 ABC (Efflux) Transporters

An experiment was done to determine if belvarafenib is a substrate of the efflux transporters P-gp and BCRP expressed on the blood-brain barrier.

Stock solutions of belvarafenib 2HCl (10 mM, 1.33 mM, 1 mM and 0.1 mM) were prepared in DMSO. Serial dilutions (7-step, 3-fold) were prepared in DMSO, and used as the test solutions in the inhibition assays (100-fold dilution). The dilution factor in substrate experiments was 100-fold. The solvent concentration in the assay buffer did not exceed 1.5% (v/v) in the assays.

Instruments used for detection include a Thermo Scientific Dionex UltiMate 3000 series UHPLC (Thermo Scientific, San Jose, CA) with a Thermo Scientific TSQ Quantum Access Max triple quadrupole MS; a Perkin Elmer MicroBeta2liquid scintillation counter (Perkin Elmer, Waltham MA) and a BMG Labtech FluoStar Omega multifunctional microplate reader (BMG Labtech, Offenburg, Germany).

Vesicular transport assays were performed with inside-out membrane vesicles prepared from cells overexpressing human ABC transporters. The transporter has been expressed by SOLVO Biotechnology in mammalian (K and M) cells by chemical selection.

Parameters of the vesicular transport assays are listed in Table 6 where: “BCRP” refers to breast cancer resistance protein; “MDR1” refers to multidrug resistance protein 1; “E3S” refers to Estrone-3-sulfate; and “NMQ” refers to N-methyl quinidine.

TABLE 5
Vesicular transport assay parameters

| | Protein content/ | Incubation | Probe | Reference |
|---|---|---|---|---|
| Transporter | Well (μg) | Time (min) | Substrate | Inhibitors |
| Human BCRP | 25 | 1 | E3S | Ko134 |
| (ABCG2) | | | (1 μM) | (1 μM) |
| Human MDR1 | 50 | 3 | NMQ | Verapamil |
| (ABCB1/P-gp) | | | (2 μM) | (100 μM) |

Belvarafenib 2HCl was incubated with membrane vesicle preparations (total protein: 50 μg/well for MDR1, and 25 g/well for BCRP) and the probe substrate. Incubations were carried out in the presence of 4 mM ATP or AMP to distinguish between transporter-mediated uptake and passive diffusion into the vesicles. Belvarafenib 2HCl was added to the reaction mixture in 0.75 μL of solvent (10 of the final incubation volume). Reaction mixtures were pre-incubated for 15 minutes at 37±1° C. (or 32±1° C. for BCRP). Reactions were initiated by the addition of 25 μL of 12 mM MgATP (or 12 mM AMP in assay buffer as a background control), preincubated separately. Reactions were quenched by the addition of 200 μL of ice-cold washing buffer and immediate filtration via glass fiber filters mounted to a 96-well plate (filter plate). The filters were washed (5×200 μL of ice-cold washing buffer), dried and the amount of substrate inside the filtered vesicles was determined by liquid scintillation counting. Treatment groups are listed in Table 6.

TABLE 6
Treatment groups in the 96-well plate format No. of wells

| | No. of |
|---|---|
| Treatment groups in the 96-well plate format | wells |
| Belvarafenib 2HCl in the respective vehicular transport (VT) | 3 |
| buffer with 1% DMSO (0.02, 0.05, 0.16, 0.49, 1.48, 4.43 and | |
| 13.3 μM) with ATP | |
| Belvarafenib 2HCl in the respective VT buffer with 1% DMSO | 3 |
| (0.02, 0.05, 0.16, 0.49, 1.48, 4.43 and 13.3 μM) with AMP | |
| 1% DMSO control with ATP | 3 |
| 1% DMSO control with AMP | 3 |
| Reference inhibitor in the respective VT buffer with 1% DMSO | 3 |
| with ATP | |
| Reference inhibitor in the respective VT buffer with 1% DMSO | 3 |
| with AMP | |

The controls were as follows. Incubation with AMP provided background activity values for all data points. Incubation with probe substrate (solvent only) provided 100% activity values. A reference inhibitor served as positive control for inhibition.

Experimental method for vesicular transport substrate assay. The uptake of belvarafenib 2HCl into membrane vesicles was determined using inside-out membrane vesicles (total protein: 50 μg/well for MDR1, or 25 μg/well for BCRP) prepared from cells overexpressing BCRP or MDR1 ABC transporters as well as from control cells. Two incubation time points (2 and 20 min) and two concentrations (1 and 10 μM) of belvarafenib 2HCl were tested in the presence of ATP or AMP, to determine whether belvarafenib 2HCl is actively transported into the vesicles. Reaction mixtures as well as start reagents (MgATP and AMP solutions) were pre-incubated for 15 minutes at 32° C. for BCRP, and 37° C. for MDR1 assay. Reactions were initiated by addition of the start reagent (MgATP or AMP) to the appropriate wells. Reactions were quenched by the addition of 200 μL of ice-cold washing buffer and immediate filtration via glass fiber filters mounted to a 96-well plate (filter plate). The filters were washed 5×200 μL of ice-cold washing buffer and dried. The amount of accumulated belvarafenib 2HCl retained inside the vesicles was determined by LC-MS/MS detection after washing the vesicles 2 times with 100 μL MeOH:H2O (2:1). Conditions and experimental groups for feasibility testing are listed in Table 7.

TABLE 7

| | No. of |
|---|---|
| Treatment groups in the 96-well plate format | wells |
| Belvarafenib 2HCl at 1 and 10 μM and two incubation times | 24 |
| (2 and 20 min) in triplicate in transporter expressing vesicles, | |
| with ATP or AMP | |
| Belvarafenib 2HCl at 1 and 10 μM and two incubation times | 24 |
| (2 and 20 min) in triplicate in control vesicles, with ATP | |
| or AMP | |
| Initial concentration values in triplicate for transporter | 12 |
| expressing and control vesicles | |

For all wells the amount of the translocated probe substrate was determined in cpm. Relative activities were calculated using the following equation: Relative Activity %=(A−B)/(C−D)*100. A is the amount of translocated substrate in the presence of TA and ATP. B is the amount of translocated substrate in the presence of TA and AMP. C is the amount of translocated substrate in the presence of solvent and ATP. D is the amount of translocated substrate in the presence of solvent and AMP.

In the vesicular transport substrate assays, the ATP-dependent transport of the TA as well as the ATP-dependent fold accumulation was calculated for each concentration and time point using the following equation, in both the transporter-containing and control vesicles:

ATP−dependent transport=n_{ATP}−N_{AMP} Fold accumulation=n_{ATP}/nAMP

n_{ATP} is the amount of translocated TA in the presence of 4 mM ATP, in pmol/mg. n_{AMP} is the amount of translocated TA in the presence of 4 mM AMP, in pmol/m.

If the ATP-dependent fold accumulation value is >2 in transporter containing vesicles and can be inhibited by a known inhibitor of the transporter, then the TA can be considered a substrate of the transporter investigated. In addition, similar ATP-dependent fold accumulation is not observed in the control vesicles.

Microsoft Excel 2010 (Microsoft Corporation, Redmond, WA) was used for basic data processing and GraphPad Prism 5.0 (GraphPad Software Inc., San Diego, CA) was used for curve fitting and determination of reaction parameters. In vesicular transport inhibition assays, the IC₅₀ (μM) was calculated, where applicable. IC₅₀ was defined as the concentration of TA required to inhibit maximal activity by 50%. IC₅₀ values were derived from a four parametric logistic equation [log(inhibitor) vs. response−variable slope]; the curve was fitted to the relative activity vs. TA concentration plot using non-linear regression. Top (maximal response) and Bottom (maximally inhibited response) values were not constrained to constant values of 100 and 0, respectively, unless it is noted otherwise.

The results indicate that belvarafenib 2HCl inhibited the BCRP-mediated E3S accumulation at the applied concentrations in a dose-dependent manner. An inhibition of 70% was detected even at the lowest investigated concentration (0.02 μM). Constraining the top of the curve at 100% an IC₅₀ value of 0.011 μM was estimated.

The results further indicate that belvarafenib 2HCl inhibited the MDR1-mediated NMQ accumulation at the applied concentrations in a dose-dependent manner with a maximum inhibition of 32%. Even though the inhibition did not reach 50%, an IC50 value of 29.64 μM was estimated for the interaction. For the estimation, a bottom constrain (0%) was applied.

In the vesicular transport substrate assays, the ATP-dependent accumulation of belvarafenib 2HCl was similar in the presence of ATP and AMP (ATP-dependent fold accumulation were <2), indicating no active accumulation of belvarafenib 2HCl in either transporter expressing or control vesicles (for both BCRP and MDR1), under all conditions tested. The positive control experiments confirmed the function of the transporter in the applied vesicles. A summary of the results is presented in Table 8.

TABLE 8

| | | | Substrate Results | |
|---|---|---|---|---|
| Inhibition Results | | | Maximum ATP | |
| | Maximum | | dependent fold | |
| Transporter | inhibition | | accumulation | |
| and assay | (% of | IC₅₀ | in transporter | Sub- |
| type | control) | (μM) | expressing vesicles | strate |
| BCRP VT | 99.8% | 0.011 | 1.51 | Unlikely |
| MDR1 VT | 32% | 29.64 | 1.23 | Unlikely |

The data show that belvarafenib 2HCl inhibited BCRP with an estimated IC₅₀ value of 0.011 μM. The data further show that Belvarafenib 2HCl inhibited MDR1 by 32% at the highest applied concentration (13.3 μM), with an estimated IC₅₀ value of 29.64 μM. In conclusion, belvarafenib 2HCl is probably not a substrate of MDR1 or BCRP as no ATP or transporter dependent accumulation was detected under the applied experimental conditions.

### Example 4: Anti-Tumor Efficacy Evaluation of Belvarafenib in Xenographic Brain Metastasis Mouse Model

The mouse strain was BALB/c Nude (nu/nu) produced and supplied by Orient Bio Inc., Korea. The mice were male and were 7 weeks of age at the start of dosing, and had a body weight range of 13.9 to 20.4 grams.

The mice were kept in conventional animal lab cages for at least one week for acclimation before the start of the experiment. The cages were polysulfone 1291H (W425×D266×H185 mm, Techniplast, Italy). Eight mice were housed in each cage at a temperature of 22±2° C., a relative humidity of 50±20%, a ventilation frequency of 10-15 times/h, a 12 hour light/dark cycle, a light intensity of 150-300 Lux, at least weekly cage replacement. The mice were fed ad libitum Picolab Rodent diet (5053, Lab Diet, USA). Tap water was given ad libitum following UV irradiation and filtration.

The cancer cell line was A375SM-Luc harboring the BRAF^{V600} mutation. That cell line was deemed suitable for the efficacy study of RAF inhibitors against BRAF^{V600} in melanoma.

The A375SM-Luc cell line was established as follows. A375SM cells were seeded into 24 well clear flat-bottomed plates with 2.5×10⁵ cells/well in MEM medium (Gibco, USA) supplemented with 10% FBS (Gibco, USA). The next day, CMV-Firefly luciferase lentivirus (Cellomics Inc., USA) was diluted in complete medium containing 8 μg/mL polybrene (Sigma Aldrich, St. Louis MO, USA). Cells were centrifuged in the prepared medium for 90 minutes at 800×g, followed by incubation in fresh medium for 2 to 3 days. Stable clones were selected using puromycin, and individual clones were screened for luciferase activity be measuring their light emission with the IVIS® Lumina Series III In Vivo Imaging System (PerkinElmer Inc., USA) after adding 10 μL (15 mg/mL) D-Luciferin (Gold Biotechnology, USA). The in vitro media was MEM 10% FBS. The in vitro culture condition was incubation at 5% CO₂ at 37° C.

The in vivo cell inoculation was as follows. Mice were anesthetized by intraperitoneal (i.p.) injection of 30 mg/kg Zoletil 50 (Virbac, France) and 10 mg/kg Rompun (Bayer Korea, Korea). Five thousand cells suspended in 10 μL phosphate buffered saline were intracranially injected into the right frontal hemisphere (AP+2.0, ML−1.0, DV+2.0 from the bregma) using a stereotactic fixation device (David Kopf Instruments, USA). Mouse models were produced over two days.

The mice were randomized by their tumor burden by BLI (8 mice per group) after 6-7 days from inoculation (implantation). The mice were then dosed with belvarafenib dihydrochloride, 99.6% purity, that was stored at room temperature. Dosing was based on active ingredient free base and was corrected for assay and water content. The dosing vehicle was DMSO (5%), cremophore EL (5%), and deionized water (90%). Belvarafenib for dosing was dissolved in the vehicle.

Belvarafenib (in groups 2 and 3) and vehicle (in group 1) were dosed at 10 mL/kg (p.o.) once per day (QD) for a duration of 102 days. The study group design and dose level are shown in Table 9 below.

TABLE 9

| Group | Individuals | Belvarafenib Dose (mg/kg) |
|---|---|---|
| 1 | 1 to 8 | Vehicle (0 mg/kg) |
| 2 | 9 to 16 | 5 mg/kg |
| 3 | 17 to 24 | 15 mg/kg |

General clinical signs were observed at least once per day during the test period. Body weight measurement was performed twice a week during the dosing period. Relative body weight (%) was calculated by: body weight Day X (g)/initial body weight (g)×100 (Day X=body weight measurement day). Tumor burden was measured by bioluminescence imaging (BLI) using a IVIS® Lumina Series III In Vivo Imaging System (PerkinElmer Inc., USA) each week. The exposure time and pixel binning were optimized in Living Image® software and the bioluminescent signal was displayed as an intensity map. Kaplan-Meier survival curves showing prolonged survival of mice of groups 1 to 3 were plotted from the date of drug administration. The median overall survival rate (mOS) was the amount of time after which 50% of the mice had died and 50% had survived.

The data were expressed as means±standard error of the mean (SEM). Statistical analysis was conducted by GraphPad PRISM® Version 6 (GraphPad Software, USA). Long-range test was conducted to evaluate significant differences between the groups. A p value less than 0.05 was considered statistically significant.

Clinical signs, body weight loss, and clinical signs are reported in Table 10 below. In belvarafenib groups 2 and 3, hair growth showed from day 7 of administration, and no other clinical signs or body weight loss were noted for 11 days. After day 11, the vehicle group showed body weight loss and a variety of clinical signs such as emaciation, hypothermia, paleness, petechial, scab, weak and abdominal distension, which were caused by debility due to tumor growth. Similar clinical signs occurred in group 2 (5 mg/kg belvarafenib) from day 14 and in group 3 (15 mg/kg belvarafenib) from day 16. The effects in groups 2 and 3 were due to the effect of the tumor, and not the drug. In Table 10: “belv.” refers to belvarafenib; “- - - -” refers to no remarkable findings; “EM” refers to emaciation; “H” refers to hypothermia; “HG” refers to hair growth; “PA” refers to paleness; “W” refers to weak; “D” refers to death; “P” refers to petechia; “S” refers to scab; and the data in the parentheses refers to the number of animals with symptoms per number of total survival animals.

TABLE 10
Clinical signs in A375SM_Luc cell brain metastasis model.

| | Vehicle | 5 mg/kg belv. | 15 mg/kg belv. |
|---|---|---|---|
| Day | (Group 1) | (Group 2) | (Group 3) |
| 1 | — | — | — |
| 2 | — | — | — |
| 3 | — | — | — |
| 4 | — | — | — |
| 5 | — | — | — |
| 6 | — | — | — |
| 7 | — | HG (5/8) | HG (4/8) |
| 8 | — | HG (8/8) | HG (8/8) |
| 9 | — | HG (8/8) | HG (8/8) |
| 10 | — | HG (8/8) | HG (8/8) |
| 11 | — | HG (8/8) | HG (8/8) |
| 12 | EM (1/8) | HG (8/8) | HG (8/8) |
| 13 | EM (2/8); PA (1/8) | HG (8/8) | HG (8/8) |
| 14 | EM (2/8); PA (1/8); | HG (8/8); EM (1/8) | HG (8/8) |
| | H (1/8); W (1/8) | | |
| 15 | D (1/8); EM (1/8); | HG (8/8); EM (1/8) | HG (8/8) |
| | W (1/8) | | |
| 16 | P (1/7); EM (1/7); | HG (7/8); EM (1/8); | HG (8/8); EM (1/8) |
| | W (1/7) | D (1/8) | |
| 17 | P (1/7); EM (1/7); | HG (7/7); EM (1/7); | HG (8/8); EM (1/8) |
| | W (1/7); S (1/7) | W (1/7); H (1/7) | |
| 18 | P (1/7); EM (1/7); | HG (6/7); D (1/7) | HG (8/8); EM (1/8); |
| | W (1/7); S (1/7); | | W (1/8) |
| | AD (1/7); H (1/7) | | |
| 19 | P (1/7); S (4/7); | HG (6/6) | HG (8/8); EM (1/8); |
| | AD (1/7); D (1/7) | | W (1/8) |
| 20 | EM (4/6); S (5/6); | HG (6/6) | HG (7/8); D (1/8) |
| | D (1/6) | | |
| 21 | S (5/5); EM (4/5) | HG (6/6) | HG (7/7) |
| 22 | S (5/5); EM (4/5); | HG (6/6) | HG (7/7) |
| | W (3/5) | | |
| 23 | S (5/5); EM (5/5); | HG (6/6); EM (1/6) | HG (7/7) |
| | W (4/5); H (1/5) | | |
| 24 | S (4/5); EM (4/5); | HG (6/6); EM (1/6) | HG (7/7) |
| | W (4/5); H (2/5); | | |
| | D (1/5) | | |
| 25 | S (2/4); EM (2/4); | HG (6/6); EM (1/6) | HG (7/7) |
| | W (2/4); H (2/4); | | |
| | D (2/4) | | |
| 26 | S (1/2); EM (1/2); | HG (6/6); EM (1/6) | HG (7/7) |
| | W (1/2); H (1/2); | | |
| | D (1/2) | | |
| 27 | S (1/1); EM (1/1); | HG (6/6); EM (1/6) | HG (7/7) |
| | W (1/1); H (1/1) | | |
| 28 | D (1/1) | HG (6/6); EM (2/6) | HG (7/7) |
| 29 | N/A | HG (6/6); EM (4/6) | HG (7/7) |
| 30 | N/A | HG (6/6); EM (4/6) | HG (7/7) |
| 31 | N/A | HG (6/6); EM (4/6); | HG (7/7) |
| | | W (2/6) | |
| 32 | N/A | HG (6/6); EM (4/6); | HG (7/7) |
| | | W (2/6); H (1/6); PA (1/6) | |
| 33 | N/A | HG (5/6); EM (5/6); | HG (7/7) |
| | | W (2/6); H (1/6); | |
| | | PA (1/6); D (1/6) | |
| 34 | N/A | HG (4/5); EM (4/5); | HG (7/7) |
| | | W (2/5); PA (1/5); D (1/5) | |
| 35 | N/A | HG (3/4); EM (3/4); | HG (7/7) |
| | | W (1/4); PA (1/4); D (1/4) | |
| 36 | N/A | HG (2/3); EM (2/3); | HG (7/7) |
| | | D (1/3) | |
| 37 | N/A | HG (2/2); EM (2/2); | HG (7/7) |
| | | W (1/2) | |
| 38 | N/A | HG (2/2); EM (2/2); | HG (7/7) |
| | | W (2/2) | |
| 39 | N/A | HG (2/2); EM (2/2); | HG (7/7) |
| | | W (2/2); PA (1/2) | |
| 40 | N/A | D (2/2) | HG (7/7) |
| 41 | N/A | N/A | HG (7/7) |
| 42 | N/A | N/A | HG (7/7) |
| 43 | N/A | N/A | HG (7/7) |
| 44 | N/A | N/A | HG (7/7) |
| 45 | N/A | N/A | HG (7/7) |
| 46 | N/A | N/A | HG (7/7) |
| 47 | N/A | N/A | HG (7/7) |
| 48 | N/A | N/A | HG (7/7); EM (1/7) |
| 49 | N/A | N/A | HG (7/7); EM (1/7) |
| 50 | N/A | N/A | HG (7/7); EM (1/7) |
| 51 | N/A | N/A | HG (7/7); EM (2/7); |
| | | | W (1/7) |
| 52 | N/A | N/A | HG (6/7); EM (1/7); |
| | | | W (1/7); D (1/7) |
| 53 | N/A | N/A | HG (6/6); EM (2/6); |
| | | | W (1/6) |
| 54 | N/A | N/A | HG (5/6); EM (1/6); |
| | | | D (1/6) |
| 55 | N/A | N/A | HG (5/5); EM (1/5) |
| 56 | N/A | N/A | HG (5/5); EM (1/5); |
| | | | W (1/5) |
| 57 | N/A | N/A | HG (5/5); EM (1/5); |
| | | | W (1/5) |
| 58 | N/A | N/A | HG (5/5); EM (1/5); |
| | | | W (1/5) |
| 59 | N/A | N/A | HG (5/5); EM (1/5); |
| | | | W (1/5) |
| 60 | N/A | N/A | HG (5/5); EM (1/5); |
| | | | W (1/5) |
| 61 | N/A | N/A | HG (4/5); D (1/5) |
| 62 | N/A | N/A | HG (4/4) |
| 63 | N/A | N/A | HG (4/4) |
| 64 | N/A | N/A | HG (4/4) |
| 65 | N/A | N/A | HG (4/4) |
| 66 | N/A | N/A | HG (4/4) |
| 67 | N/A | N/A | HG (4/4) |
| 68 | N/A | N/A | HG (4/4) |
| 69 | N/A | N/A | HG (4/4) |
| 70 | N/A | N/A | HG (4/4) |
| 71 | N/A | N/A | HG (4/4) |
| 72 | N/A | N/A | HG (4/4) |
| 73 | N/A | N/A | HG (4/4) |
| 74 | N/A | N/A | HG (4/4) |
| 75 | N/A | N/A | HG (4/4); AD (1/4) |
| 76 | N/A | N/A | HG (4/4); AD (1/4) |
| 77 | N/A | N/A | HG (4/4); AD (1/4) |
| 78 | N/A | N/A | HG (4/4); AD (1/4); |
| | | | S (1/4) |
| 79 | N/A | N/A | HG (3/4); D (1/4) |
| 80 | N/A | N/A | HG (3/3) |
| 81 | N/A | N/A | HG (3/3) |
| 82 | N/A | N/A | HG (3/3) |
| 83 | N/A | N/A | HG (3/3) |
| 84 | N/A | N/A | HG (3/3) |
| 85 | N/A | N/A | HG (3/3); EM (1/3) |
| 86 | N/A | N/A | HG (3/3); EM (1/3) |
| 87 | N/A | N/A | HG (3/3); EM (1/3) |
| 88 | N/A | N/A | HG (3/3); EM (1/3); |
| | | | H (1/3) |
| 89 | N/A | N/A | HG (3/3); EM (1/3); |
| | | | H (1/3) |
| 90 | N/A | N/A | HG (3/3); EM (1/3); |
| | | | H(1/3) |
| 91 | N/A | N/A | HG (3/3); EM (1/3); |
| | | | H(1/3) |
| 92 | N/A | N/A | HG (3/3); EM (1/3); |
| | | | H(1/3) |
| 93 | N/A | N/A | HG (2/3); EM (1/3); |
| | | | H (1/3); W (1/3) |
| 94 | N/A | N/A | HG (2/3); EM (1/3); |
| | | | H(1/3); W (1/3) |
| 95 | N/A | N/A | HG (2/3); EM (1/3); |
| | | | H (1/3); W (1/3) |
| 96 | N/A | N/A | HG (2/3); EM (1/3); |
| | | | H (1/3); W (1/3) |
| 97 | N/A | N/A | HG (2/3); EM (1/3); |
| | | | H (1/3); W (1/3) |
| 98 | N/A | N/A | HG (2/3); EM (1/3); |
| | | | H (1/3); W (1/3) |
| 99 | N/A | N/A | HG (2/3); D (1/3) |
| 100 | N/A | N/A | HG (2/2) |
| 101 | N/A | N/A | HG (2/2) |
| 102 | N/A | N/A | HG (2/2) |
| 103 | N/A | N/A | HG (2/2) |

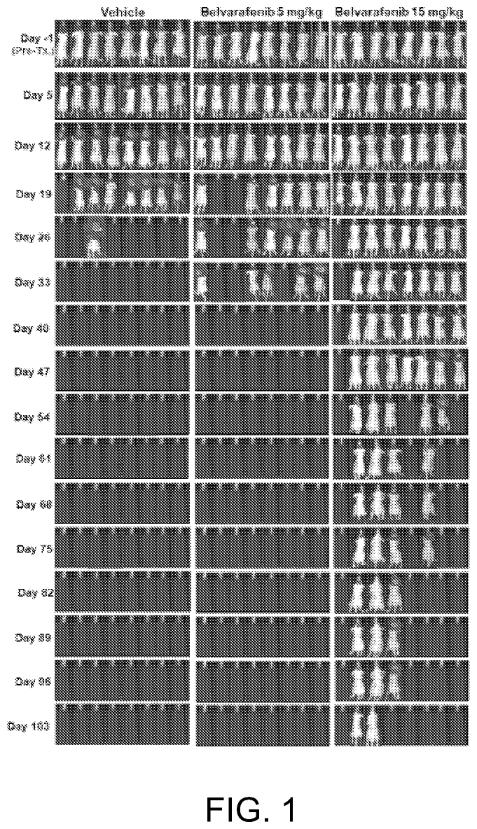

Mice were intraperitoneally injected with 15 mg/mL of D-Luciferin on each of days 1, 5, 12, 19, 26, 33, 40, 47, 54, 61, 68, 75, 82, 89, 96, and 103 and evaluated with BLI imaging with an IVIS® Lumina Series III In Vivo Image System (PerkinElmer Inc., USA). The results are presented in FIG. 1.

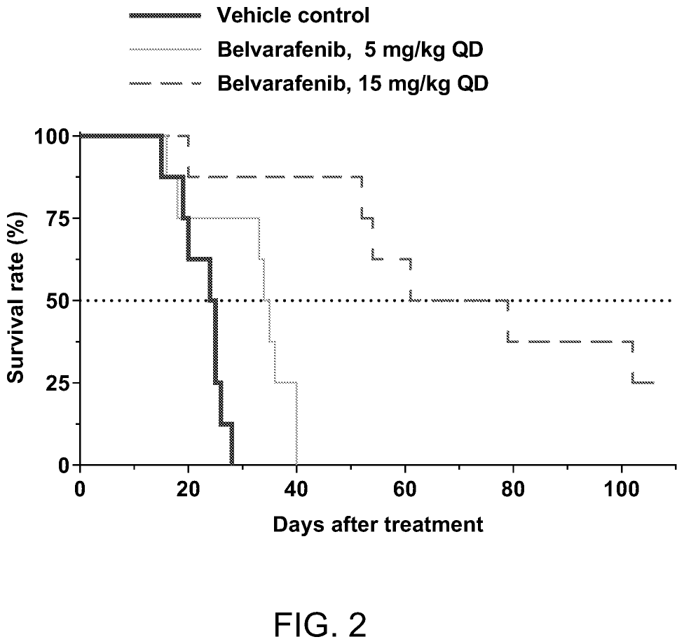

BLI was used to quantify longitudinal brain growth. Belvarafenib dosed at 5 mg/kg showed more effective tumor inhibition than the vehicle, and belvarafenib dosed at 15 mg/kg led to a markedly delayed tumor growth as compared to the vehicle. More particularly, belvarafenib dosed at 5 mg/kg and 15 mg/kg resulted in a significantly prolonged survival with a median survival time (mOS) of 34.5 days (p<0.05) and 70.0 days (p<0.001), respectively, compared to the vehicle (mOS 24.5 days). See FIG. 2 and Table 11. In Table 11: “mOS” refers to median overall survival; and maximum weight loss was calculated by (1−mean of individual relative body weight)×100 in day Y. The day Y has a maximum body weight loss for the example.

TABLE 11

| Treatment | mOS (days) | Maximum weight loss (%) |
|---|---|---|
| Vehicle | 24.5 | 22.2 (day 25) |
| Belvarafenib (5 mg/kg) | 34.5 | 33.0 (day 39) |
| Belvarafenib (15 mg/kg) | 70.0 | — |

The raw data for Table 11 is presented in Tables 12 to 14 for the vehicle (Table 12), 5 mg/kg belvarafenib (Table 13), and 15 mg/kg belvarafenib (Table 14).

TABLE 12
Individual body weight in grams for A375SM_Luc
cell brain metastasis mouse model for the vehicle

| | Day | | | | | | | | | |
|---|---|---|---|---|---|---|---|---|---|---|
| Animal | 1 | 2 | 3 | 4 | 5 | 6 | 7 | 8 | MEAN | SEM |
| 0 | 17.3 | 18.2 | 17.3 | 20.3 | 17.1 | 17.8 | 19.6 | 13.9 | 17.7 | 0.7 |
| 4 | 18.0 | 19.2 | 17.9 | 20.3 | 17.3 | 18.3 | 19.4 | 13.6 | 18.0 | 0.7 |
| 8 | 17.9 | 19.1 | 18.2 | 20.4 | 18.0 | 19.4 | 20.4 | 12.8 | 18.3 | 0.9 |
| 11 | 17.2 | 18.5 | 17.9 | 20.0 | 18.0 | 19.0 | 20.5 | 12.0 | 17.9 | 0.9 |
| 15 | 15.9 | 17.3 | 17.2 | 20.0 | 16.8 | 18.5 | 18.9 | 10.5 | 16.9 | 1.0 |
| 18 | — | 16.0 | 16.7 | 18.7 | 15.7 | 17.9 | 18.0 | 9.8 | 16.1 | 1.1 |
| 22 | — | 13.0 | 15.3 | — | 14.3 | 14.7 | 16.5 | — | 14.8 | 0.6 |
| 25 | — | — | 15.0 | — | — | — | 13.5 | — | 14.3 | 0.8 |

TABLE 13
Individual body weight in grams for A375SM_Luc cell
brain metastasis mouse model for 5 mg/kg Belvarafenib

| | Day | | | | | | | | | |
|---|---|---|---|---|---|---|---|---|---|---|
| Animal | 9 | 10 | 11 | 12 | 13 | 14 | 15 | 16 | MEAN | SEM |
| 0 | 15.6 | 17.1 | 18.1 | 18.7 | 17.8 | 17.4 | 17.9 | 19.6 | 17.8 | 0.4 |
| 4 | 14.9 | 17.6 | 15.8 | 19.3 | 18.3 | 17.5 | 18.2 | 19.6 | 17.7 | 0.6 |
| 8 | 15.1 | 17.4 | 16.2 | 19.6 | 18.6 | 17.5 | 18.6 | 20.3 | 17.9 | 0.6 |
| 11 | 15.0 | 18.0 | 17.7 | 19.4 | 18.7 | 17.6 | 18.6 | 20.5 | 18.2 | 0.6 |
| 15 | 15.6 | 13.5 | 12.6 | 19.2 | 18.8 | 17.8 | 19.3 | 21.1 | 17.2 | 1.1 |
| 18 | 16.6 | — | — | 19.4 | 19.2 | 19.1 | 19.4 | 21.6 | 19.2 | 0.6 |
| 22 | 16.1 | — | — | 18.4 | 19.0 | 19.4 | 19.3 | 21.6 | 19.0 | 0.7 |
| 25 | 16.6 | — | — | 16.8 | 12.6 | 19.4 | 19.5 | 21.0 | 17.7 | 1.2 |
| 29 | 15.8 | — | — | 14.3 | 15.3 | 18.7 | 17.7 | 18.2 | 16.7 | 0.7 |
| 32 | 14.9 | — | — | 13.0 | 13.6 | 13.2 | 15.5 | 15.8 | 14.3 | 0.5 |
| 36 | 13.9 | — | — | — | — | — | — | 14.4 | 14.2 | 0.3 |
| 39 | 11.2 | — | — | — | — | — | — | 12.2 | 11.7 | 0.5 |

TABLE 14
Individual body weight in grams for A375SM_Luc cell
brain metastasis mouse model for 5 mg/kg belvarafenib

| | Day | | | | | | | | | |
|---|---|---|---|---|---|---|---|---|---|---|
| Animal | 17 | 18 | 19 | 20 | 21 | 22 | 23 | 24 | MEAN | SEM |
| 0 | 17.1 | 19.5 | 20.4 | 16.7 | 17.4 | 17.9 | 17.1 | 19.8 | 18.2 | 0.5 |
| 4 | 17.4 | 19.3 | 20.3 | 17.1 | 17.4 | 18.3 | 17.5 | 19.8 | 18.4 | 0.4 |
| 8 | 17.8 | 19.8 | 20.8 | 18.6 | 18.0 | 18.7 | 17.8 | 20.3 | 19.0 | 0.4 |
| 11 | 17.9 | 20.2 | 20.9 | 18.4 | 17.8 | 18.3 | 18.0 | 20.8 | 19.0 | 0.5 |
| 15 | 16.8 | 20.2 | 21.5 | 18.3 | 17.9 | 19.0 | 18.0 | 20.6 | 19.0 | 0.6 |
| 18 | 12.9 | 20.9 | 21.9 | 18.9 | 18.5 | 19.1 | 18.9 | 21.5 | 19.1 | 1.0 |
| 22 | — | 21.0 | 22.1 | 18.7 | 18.5 | 20.1 | 19.3 | 21.0 | 20.1 | 0.5 |
| 25 | — | 21.7 | 22.6 | 19.2 | 19.6 | 20.3 | 19.4 | 21.4 | 20.6 | 0.5 |
| 29 | — | 21.3 | 22.2 | 19.9 | 19.6 | 20.3 | 19.5 | 21.9 | 20.7 | 0.4 |
| 32 | — | 21.6 | 23.0 | 19.8 | 19.5 | 20.2 | 19.1 | 22.0 | 20.7 | 0.6 |
| 36 | — | 21.1 | 23.3 | 19.9 | 19.8 | 20.5 | 19.5 | 21.6 | 20.8 | 0.5 |
| 39 | — | 21.6 | 23.4 | 20.0 | 19.5 | 20.6 | 19.8 | 21.9 | 21.0 | 0.5 |
| 42 | — | 21.1 | 22.8 | 19.7 | 19.4 | 20.6 | 19.3 | 20.7 | 20.5 | 0.5 |
| 46 | — | 21.9 | 23.1 | 20.5 | 19.7 | 21.1 | 19.4 | 18.4 | 20.6 | 0.6 |
| 50 | — | 21.8 | 23.1 | 20.1 | 20.3 | 21.0 | 18.4 | 14.0 | 19.8 | 1.1 |
| 53 | — | 22.0 | 23.1 | 20.6 | 20.5 | 21.3 | 16.3 | — | 20.6 | 1.0 |
| 57 | — | 21.3 | 23.2 | 19.8 | — | 20.6 | 12.1 | — | 19.4 | 1.9 |
| 60 | — | 20.9 | 23.4 | 19.8 | — | 20.3 | 11.5 | — | 19.2 | 2.0 |
| 64 | — | 21.4 | 23.1 | 20.6 | — | 21.0 | — | — | 21.5 | 0.5 |
| 68 | — | 22.1 | 24.1 | 20.8 | — | 21.7 | — | — | 22.2 | 0.7 |
| 71 | — | 21.9 | 23.9 | 20.4 | — | 20.5 | — | — | 21.7 | 0.8 |
| 74 | — | 22.2 | 24.4 | 20.2 | — | 21.0 | — | — | 22.0 | 0.9 |
| 78 | — | 22.0 | 24.2 | 19.8 | — | 21.6 | — | — | 21.9 | 0.9 |
| 81 | — | 22.4 | 24.3 | 19.8 | — | — | — | — | 22.2 | 1.3 |
| 85 | — | 22.1 | 24.1 | 17.6 | — | — | — | — | 21.3 | 1.9 |
| 88 | — | 22.5 | 24.7 | 16.6 | — | — | — | — | 21.3 | 2.4 |
| 92 | — | 21.6 | 24.1 | 14.5 | — | — | — | — | 20.1 | 2.9 |
| 95 | — | 21.5 | 24.2 | 13.3 | — | — | — | — | 19.7 | 3.3 |
| 99 | — | 21.3 | 23.8 | — | — | — | — | — | 22.6 | 1.3 |
| 102 | — | 21.1 | 25.3 | — | — | — | — | — | 23.2 | 2.1 |

### Example 5: Brain to Plasma Ratio of Belvarafenib as Compared to Prior Art Pan-RAF, BRAF V600E and MEK Inhibitors

The brain to plasma ratio of the pan-RAF inhibitor belvarafenib was compared to: the pan-RAF inhibitor DAY101 (tovorafenib, MLN2480); the BRAF V^{600E} inhibitors dabrafenib, vemurafenib, and encorafenib; and the MEK inhibitors cobimetinib, trametinib, and selumetinib. The results are presented in Table 15 below. The brain to plasma ratio is expressed in terms of B/P (nonclinical) defined as the percentage of brain exposure relative to plasma. The B/P for belvarafenib was determined experimentally according to the present disclosure and wherein the B/P of 93% is for mice and the B/P of 138% is for rats. The B/P for DAY101 is for mice was taken from Gampa, et al., “Brain Distribution and Active Efflux of Three pan-RAF Inhibitors: Considerations in the Treatment of Melanoma Brain Metastases”, J Pharmacol Exp Ther 368:446-461, March 2019. The B/P for dabrafenib is for mice and was taken from Mittapalli, et al., “Mechanisms Limiting Distribution of the Threonine-Protein Kinase B-RaFV600E Inhibitor Dabrafenib to the Brain: Implications for the Treatment of Melanoma Brain Metastases”, J Pharmacol Exp Ther 344:655-364, 2013. The B/P for vemurafenib is for mice and was taken from Mittapalli, et al., “Impact of P-glycoprotein (ABCB1) and breast cancer resistance protein (ABCG2) on the brain distribution of a novel BRAF inhibitor: vemurafenib (PLX4032)”, J Pharmacol Exp Ther 342:33-40, 2013. The B/P for encorafenib is for mice and was taken from Wang, et al., “P-glycoprotein (MDR1/ABCB1) and Breast Cancer Resistance Protein (BCRP/ABCG2) affect brain accumulation and intestinal disposition of encorafenib in mice”, Pharmacol Res. 2018 March; 414-423. The B/P for cobimetinib is for mice and was taken from Choo, et al., “Role of P-glycoprotein on the brain penetration and brain pharmacodynamic activity of the MEK inhibitor cobimetinib”, Mol Pharm. 2014 Nov. 3; 11(11):4199-207. The B/P for trametinib is for mice and was taken from Vaidhyanathan, et al., “Factors Influencing the CNS Distribution of a Novel MEK-1/2 Inhibitor: Implications for Combination Therapy for Melanoma Brain Metastases”, Drug Metab Dispos. 2104 August; 42(8): 1292-1300. The B/P for selumetinib is for mice and was taken from Gooijer, et al., “The impact of P-glycoprotein and breast cancer resistance protein on the brain pharmacokinetics and pharmacodynamics of a panel of MEK inhibitors”, Int J Cancer. 2018 Jan. 15; 142(2):381-391. In Table 15, “P-gp” refers to P-glycoprotein (MDR1, ABDB1), and “BCRP” refers to breast cancer resistance protein (ABCG2).

TABLE 15

| MEK Inhibitor | B/P | P-gp/BCRP efflux substrate? |
|---|---|---|
| Belvarafenib | 93-138% | No |
| DAY101 | 20-34% | Yes (BCRP) |
| Dabrafenib | 2-4% | Yes (P-gp + BCRP) |
| Vemurafenib | 0.4-1% | Yes (P-gp + BCRP) |
| Encorafenib | 0.4% | Yes (P-gp + BCRP) |
| Cobimetinib | 30% | Yes (P-gp) |
| Trametinib | 15% | Yes (P-gp + BCRP) |
| Selumetinib | 2% | Yes (P-gp + BCRP) |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of treating metastatic melanoma comprising administering to a subject in need thereof an amount of belvarafenib effective to treat the metastatic melanoma, wherein the site of metastasis is within the subject's brain.

2. The method of claim 1, wherein the melanoma carries a RAF mutation.

3. The method of claim 2, wherein the melanoma carries a BRAF^{V600E} mutation.

4. The method of any one of claims 1 to 3, wherein the melanoma carries a NRAS mutation or a KRAS mutation.

5. The method of claim 4, wherein the melanoma carries a NRAS mutation.

6. The method of claim 5, wherein the melanoma carries a NRAS^{G12D} mutation, a NRAS^{Q61K} mutation, a NRAS^{Q61R} mutation, a NRAS^{G12C} mutation, a NRAS^{Q61H} mutation, a NRAS^{Q61L} mutation, and combinations thereof.

7. The method of any one of claims 1 to 6, wherein the subject is treated with from about 2.5 mg per kg body weight to about 25 mg per kg of body weight of belvarafenib, or a pharmaceutically acceptable salt thereof, per day.

8. The method of claim 7, wherein the subject is treated with about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg of belvarafenib, or a pharmaceutically acceptable salt thereof, per day.

9. The method of claim 7 or claim 8, wherein the subject is treated with about 250 mg, about 300 mg, about 350 mg, about 400 mg, about 450 mg, or about 500 mg of belvarafenib, or a pharmaceutically acceptable salt thereof, twice per day.

10. The method of any one of claims 7 to 9, wherein belvarafenib is administered daily for 28 consecutive days of a 28-day treatment cycle.

11. The method of any one of claims 1 to 10, wherein belvarafenib is administered orally.

12. A method of treating a brain cancer, the method comprising:
(i) administering to a subject in need thereof a therapeutically effective amount of belvarafenib, or a pharmaceutically acceptable salt thereof, to treat the brain cancer;
(ii) wherein administration of the belvarafenib inhibits the growth and viability of brain cancer cells in said subject; and
(iii) wherein the brain cancer is characterized by a mutated MAPK signaling pathway.

13. The method of claim 12, wherein the cancer is a selected from glioblastoma and a metastatic cancer selected from melanoma, lung, breast, colorectal (CRC), bladder, gallbladder, nephroblastoma, gastrointestinal stromal tumor (GIST), prostate, myeloid leukemia, multiple myeloma, thyroid, biliary, adenocarcinoma, choriocarcinoma, sarcoma, squamous cell, and combinations thereof.

14. The method of claim 13, wherein the metastatic cancer is selected from melanoma, nephroblastoma, GIST, CRC, sarcoma, gallbladder, bladder, and combinations thereof.

15. The method of any one of claims 12 to 14, wherein the cancer carries a RAF mutation.

16. The method of claim 15, wherein the cancer carries a BRAF^{V600E} mutation.

17. The method of claim 15 or claim 16, wherein the cancer is selected from nephroblastoma carrying a BRAF^{V600E} mutation, melanoma carrying a BRAF^{V600E} mutation, GIST carrying a BRAF^{V600E} mutation, CRC carrying a BRAF^{V600E} mutation, and combinations thereof.

18. The method of claim 17, wherein the cancer is a melanoma carrying a BRAF^{V600E} mutation, nephroblastoma carrying a BRAF^{V600E} mutation, GIST carrying a BRAF^{V600E} mutation, and combinations thereof.

19. The method of claim 18, wherein the cancer is selected from melanoma carrying a BRAF^{V600E} mutation, GIST carrying a BRAF^{V600E} mutation, and combinations thereof.

20. The method of any one of claims 16 to 19, wherein the melanoma is metastatic or unresectable.

21. The method of any one of claims 12 to 20, wherein the cancer carries a NRAS mutation or a KRAS mutation.

22. The method of claim 21, wherein the cancer has at least one mutation selected from a BRAF^{V600E} mutation, a KRAS^{G12V} mutation, a KRAS^{G12D} mutation, a KRAS^{G12C} mutation, a KRAS^{Q61H} mutation, a NRAS^{G12D} mutation, a NRAS^{Q61K} mutation, a NRAS^{Q61R} mutation, a NRAS^{Q61H} mutation, a NRAS^{Q61L} mutation, and a NRAS^{G12C} mutation.

23. The method of claim 22, wherein the cancer is selected from sarcoma carrying a KRAS^{G12V} mutation, melanoma carrying a NRAS^{G12D} mutation, melanoma carrying a NRAS^{Q61K} mutation, melanoma carrying a NRAS^{Q61R} mutation, melanoma carrying a NRAS^{Q61H} mutation melanoma carrying a NRAS^{Q61L} mutation, melanoma carrying a NRAS^{G12C} mutation, gallbladder cancer carrying a KRAS^{G12D} mutation, CRC carrying a KRAS^{G12C} mutation, CRC carrying a KRAS^{G12V} mutation, CRC carrying a KRAS^{Q61H} mutation, CRC carrying a KRAS^{G12D} mutation, bladder cancer carrying a KRA^{G12D} mutation, bladder cancer carrying a KRAS^{G12V} mutation, and combinations thereof.

24. The method of claim 23, wherein the cancer is sarcoma carrying a KRAS^{G12V} mutation, melanoma carrying a NRAS^{Q61R} mutation, melanoma carrying a NRAS^{Q61H} mutation, gallbladder cancer carrying a KRAS^{G12D} mutation, CRC carrying a KRAS^{G12C} mutation, CRC carrying a KRAS^{G12V} mutation, CRC carrying a KRAS^{G12D}mutation, bladder cancer carrying a KRAS^{G12D} mutation, bladder cancer carrying a KRAS^{G12V} mutation, and combinations thereof.

25. The method of claim 22, wherein the cancer is selected from melanoma carrying a NRAS^{Q61L} mutation, melanoma carrying a NRAS^{Q61H} mutation, melanoma carrying a NRAS^{Q61K} mutation, melanoma carrying a NRAS^{Q61R} mutation, and combinations thereof.

26. The method of any one of claims 21 to 25, wherein the cancer is melanoma carrying a NRAS mutation.

27. The method of any one of claims 12 to 26, wherein the subject is treated with from about 2.5 mg per kg body weight to about 25 mg per kg of body weight of belvarafenib, or a pharmaceutically acceptable salt thereof, per day.

28. The method of claim 27, wherein the subject is treated with about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg of belvarafenib, or a pharmaceutically acceptable salt thereof, per day.

29. The method of claim 27 or claim 28, wherein the subject is treated with about 250 mg, about 300 mg, about 350 mg, about 400 mg, about 450 mg, or about 500 mg of belvarafenib, or a pharmaceutically acceptable salt thereof, twice per day.

30. The method of any one of claims 27 to 29, wherein belvarafenib is administered daily for 28 consecutive days of a 28-day treatment cycle.

31. The method of any one of claims 12 to 30, wherein belvarafenib is administered orally.

32. The method of any one of claims 12 to 31, wherein the subject is a human.

33. The method of any one of claims 12 to 32, further comprising administering at least one additional therapy.

34. The method of claim 33, wherein the at least one additional therapy is a chemotherapeutic agent.

35. The method of claim 34, wherein the at least one additional therapy is a MEK inhibitor.

36. The method of claim 35, wherein the MEK inhibitor is cobimetinib.

37. The method of any one of claims 12 to 36, wherein the administering results in one or more of: (i) inhibition of brain cancer metastases; (ii) reduction in brain cancer metastases size; (iii) reduction in brain cancer metastases number; (iv) reduction of number of brain cancer cells; (v) reduction of brain cancer cell viability; and (vi) inhibition of brain cancer cell growth.

38. A method of treating brain cancer, the method comprising:
(i) administering to a subject in need thereof a therapeutically effective amount of belvarafenib, or a pharmaceutically acceptable salt thereof;
(ii) administering to the subject an effective amount of a MEK inhibitor, or a pharmaceutically acceptable salt thereof,
(iii) wherein administration of the belvarafenib and the MEK inhibitor inhibits the growth and viability of brain cancer cells in said subject; and
(iv) wherein the brain cancer is characterized by a mutated MAPK signaling pathway.

39. The method of claim 38, wherein the cancer is a selected from glioblastoma and a metastatic cancer selected from melanoma, lung, breast, colorectal (CRC), bladder, gallbladder, nephroblastoma, gastrointestinal stromal tumor (GIST), prostate, myeloid leukemia, multiple myeloma, thyroid, biliary, adenocarcinoma, choriocarcinoma, sarcoma, squamous cell, and combinations thereof.

40. The method of claim 39, wherein the metastatic cancer is selected from melanoma, nephroblastoma, GIST, CRC, sarcoma, gallbladder, bladder, and combinations thereof.

41. The method of any one of claims 38 to 40, wherein the cancer carries a RAF mutation.

42. The method of claim 41, wherein the cancer carries a BRAF V600E mutation.

43. The method of claim 41 or claim 42, wherein the cancer is selected from nephroblastoma carrying a BRAF^{V600E}, melanoma carrying a BRAF^{V600E} mutation, GIST carrying a BRAF^{V600E} mutation, CRC carrying a BRAF^{V600E} mutation, and combinations thereof.

44. The method of claim 43, wherein the cancer is a melanoma carrying a BRAF^{V600E} mutation, nephroblastoma carrying a BRAF^{V600E} mutation, GIST carrying a BRAF^{V600E} mutation, and combinations thereof.

45. The method of claim 44, wherein the cancer is selected from melanoma carrying a BRAF^{V600E} mutation, GIST carrying a BRAF^{V600E} mutation, and combinations thereof.

46. The method of any one of claims 42 to 45, wherein the melanoma is metastatic or unresectable.

47. The method of any one of claims 38 to 46, wherein the cancer carries a NRAS mutation or a KRAS mutation.

48. The method of claim 47, wherein the cancer has at least one mutation selected from a BRAF^{V600E} mutation, a KRAS^{G12V} mutation, a KRAS^{G12D} mutation, a KRAS^{G12C} mutation, a KRAS^{Q61H} mutation, a NRAS^{G12D} mutation, a NRAS^{Q61K} mutation, a NRAS^{Q61R} mutation, a NRAS^{Q61H} mutation, a NRAS^{Q61L} mutation, and a NRAS^{G12C} mutation.

49. The method of claim 48, wherein the cancer is selected from sarcoma carrying a KRAS^{G12V} mutation, melanoma carrying a NRAS^{G12D} mutation, melanoma carrying a NRAS^{Q61K} mutation, melanoma carrying a NRAS^{Q61R} mutation, melanoma carrying a NRAS^{Q61H} mutation, melanoma carrying a NRAS^{Q61L} mutation, melanoma carrying a NRAS^{G12C} mutation, gallbladder cancer carrying a KRAS^{G12D} mutation, CRC carrying a KRAS^{G12C} mutation, CRC carrying a KRAS^{G12V} mutation, CRC carrying a KRAS^{Q61H} mutation, CRC carrying a KRAS^{G12D} mutation, bladder cancer carrying a KRAS^{G12D} mutation, bladder cancer carrying a KRAS^{G12V} mutation, and combinations thereof.

50. The method of claim 49, wherein the cancer is sarcoma carrying a KRAS^{G12V} mutation, melanoma carrying a NRAS^{G61R} mutation, melanoma carrying a NRAS^{G61H} mutation, gallbladder cancer carrying a KRAS^{G12D} mutation, CRC carrying a KRAS^{G12C} mutation, CRC carrying a KRAS^{G12V} mutation, CRC carrying a KRAS^{G12D} mutation, bladder cancer carrying a KRAS^{G12D} mutation, bladder cancer carrying a KRAS^{G12V} mutation, and combinations thereof.

51. The method of claim 49, wherein the cancer is selected from melanoma carrying a NRAS^{G61L} mutation, melanoma carrying a NRAS^{Q61H} mutation, melanoma carrying a NRAS^{Q61K} mutation, melanoma carrying a NRAS^{Q61R} mutation, and combinations thereof.

52. The method of any one of claims 47 to 51, wherein the cancer is melanoma carrying a NRAS mutation.

53. The method of any one of claims 38 to 52, wherein the subject is treated with from about 2.5 mg per kg body weight to about 25 mg per kg of body weight of belvarafenib, or a pharmaceutically acceptable salt thereof, per day.

54. The method of claim 53, wherein the subject is treated with about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg of belvarafenib, or a pharmaceutically acceptable salt thereof, per day.

55. The method of claim 53 or claim 54, wherein the subject is treated with about 250 mg, about 300 mg, about 350 mg, about 400 mg, about 450 mg, or about 500 mg of belvarafenib, or a pharmaceutically acceptable salt thereof, twice per day.

56. The method of any one of claims 53 to 55, wherein belvarafenib is administered daily for 28 consecutive days of a 28-day treatment cycle.

57. The method of any one of claims 38 to 56, wherein belvarafenib is administered orally.

58. The method of any one of claims 38 to 57, wherein the subject is treated with from about 20 mg to about 100 mg, from about 40 mg to about 80 mg, or about 60 mg of the MEK inhibitor per day.

59. The method of any one of claims 38 to 58, wherein the MEK inhibitor is cobimetinib or a pharmaceutically acceptable salt thereof, and further wherein the subject is treated with about 60 mg, about 40 mg, or about 20 mg per day of the cobimetinib.

60. The method of any one of claims 38 to 59, wherein the MEK inhibitor is administered once daily for 21 consecutive days of a 28-day treatment cycle.

61. The method of any one of claims 38 to 60, wherein the subject is a human.

62. The method of any one of claims 38 to 61, wherein the administering results in one or more of: (i) inhibition of brain cancer metastases; (ii) reduction in brain cancer metastases size; (iii) reduction in brain cancer metastases number; (iv) reduction of number of brain cancer cells; (v) reduction of brain cancer cell viability; and (vi) inhibition of brain cancer cell growth.
